SEQUENCE LISTING (169 sequences)

SEQ ID NO: 1 - DNA, 18 bp, Artificial Sequence
  oligonucleotide block_miR-486-5p_v3a
  misc_feature (1)..(5): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (13)..(13): LNA nucleotide
  misc_feature (15)..(18): 2′-O-methyl ribonucleotide
cucggggcagctcaguac

SEQ ID NO: 2 - DNA, 18 bp, Artificial Sequence
  oligonucleotide block_miR-16-5p_v3
  misc_feature (1)..(4): 2′-O-methyl ribonucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (15)..(18): 2′-O-methyl ribonucleotide
ccaatatttacgtgcugc

SEQ ID NO: 3 - DNA, 18 bp, Artificial Sequence
  oligonucleotide block_miR-191-5p_v3
  misc_feature (1)..(4): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (15)..(18): 2′-O-methyl ribonucleotide
gcugcttttgggatuccg

SEQ ID NO: 4 - DNA, 20 bp, Artificial Sequence
  oligonucleotide block_hY4_v3a
  misc_feature (1)..(4): 2′-O-methyl ribonucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (16)..(16): LNA nucleotide
  misc_feature (17)..(20): 2′-O-methyl ribonucleotide
cagucaaatttagcaguggg

SEQ ID NO: 5 - DNA, 17 bp, Artificial Sequence
  oligonucleotide block_miR-25-3p_v3
  misc_feature (1)..(4): 2′-O-methyl ribonucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (14)..(17): 2′-O-methyl ribonucleotide
cagaccgagacaagugc

SEQ ID NO: 6 - DNA, 17 bp, Artificial Sequence
  oligonucleotide block_miR-223-3p_v3
  misc_feature (1)..(4): 2′-O-methyl ribonucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (14)..(17): 2′-O-methyl ribonucleotide
ggguatttgacaaacug

SEQ ID NO: 7 - DNA, 21 bp, Artificial Sequence
  oligonucleotide block_let-7a_v3
  misc_feature (1)..(3): 2′-O-methyl ribonucleotide
  misc_feature (4)..(4): LNA nucleotide
  misc_feature (6)..(6): LNA nucleotide
  misc_feature (8)..(8): LNA nucleotide
  misc_feature (11)..(11): LNA nucleotide
  misc_feature (18)..(21): 2′-O-methyl ribonucleotide
aactatacaacctactaccuc

SEQ ID NO: 8 - DNA, 19 bp, Artificial Sequence
  oligonucleotide block_let-7i_v3a
  misc_feature (1)..(4): 2′-O-methyl ribonucleotide
  misc_feature (5)..(5): LNA nucleotide
  misc_feature (7)..(7): LNA nucleotide
  misc_feature (10)..(10): LNA nucleotide
  misc_feature (12)..(12): LNA nucleotide
  misc_feature (14)..(14): LNA nucleotide
  misc_feature (16)..(19): 2′-O-methyl ribonucleotide
aacagcacaaactacuacc

SEQ ID NO: 9 - DNA, 18 bp, Artificial Sequence
  oligonucleotide block_miR-93-5p_v3
  misc_feature (1)..(4): 2′-O-methyl ribonucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (15)..(18): 2′-O-methyl ribonucleotide
ccugcacgaacagcacuu

SEQ ID NO: 10 - DNA, 17 bp, Artificial Sequence
  oligonucleotide block_miR-140-3p_v3
  misc_feature (1)..(4): 2′-O-methyl ribonucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (14)..(17): 2′-O-methyl ribonucleotide
guggttctaccctgugg

SEQ ID NO: 11 - DNA, 20 bp, Artificial Sequence
  oligonucleotide block_miR-30d-5p_v3
  misc_feature (1)..(4): 2′-O-methyl ribonucleotide
  misc_feature (5)..(5): LNA nucleotide
  misc_feature (7)..(7): LNA nucleotide
  misc_feature (12)..(12): LNA nucleotide
  misc_feature (14)..(14): LNA nucleotide
  misc_feature (16)..(16): LNA nucleotide
  misc_feature (17)..(20): LNA nucleotide
uccagtcggggatgttuaca

SEQ ID NO: 12 - DNA, 17 bp, Artificial Sequence
  oligonucleotide block_miR-182-5p_v3
  misc_feature (1)..(4): 2′-O-methyl ribonucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (14)..(17): 2′-O-methyl ribonucleotide
ugagttctaccatugcc

SEQ ID NO: 13 - DNA, 20 bp, Artificial Sequence
  oligonucleotide block_miR-185-5p_v3
  misc_feature (1)..(4): 2′-O-methyl ribonucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (16)..(16): LNA nucleotide
  misc_feature (17)..(20): 2′-O-methyl ribonucleotide
caggaactgcctttctcucc

SEQ ID NO: 14 - DNA, 19 bp, Artificial Sequence
  oligonucleotide block_miR-103a-3p_v3
  misc_feature (1)..(4): 2′-O-methyl ribonucleotide
  misc_feature (6)..(6): LNA nucleotide
  misc_feature (8)..(8): LNA nucleotide
  misc_feature (11)..(11): LNA nucleotide
  misc_feature (13)..(13): LNA nucleotide
  misc_feature (15)..(15): LNA nucleotide
  misc_feature (16)..(19): 2′-O-methyl ribonucleotide
cauagccctgtacaaugcu

SEQ ID NO: 15 - DNA, 21 bp, Artificial Sequence
  oligonucleotide block_let-7b_v3
  misc_feature (1)..(3): 2′-O-methyl ribonucleotide
  misc_feature (4)..(4): LNA nucleotide
  misc_feature (6)..(6): LNA nucleotide
  misc_feature (8)..(8): LNA nucleotide
  misc_feature (11)..(11): LNA nucleotide
  misc_feature (18)..(21): 2′-O-methyl ribonucleotide
aaccacacaacctactaccuc

SEQ ID NO: 16 - DNA, 19 bp, Artificial Sequence
  oligonucleotide block_miR-451a_v3
  misc_feature (1)..(5): 2′-O-methyl ribonucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (15)..(15): LNA nucleotide
  misc_feature (16)..(19): 2′-O-methyl ribonucleotide
acucagtaatggtaacggu

SEQ ID NO: 17 - DNA, 19 bp, Artificial Sequence
  oligonucleotide block_miR-150-5p_v3
  misc_feature (1)..(5): 2′-O-methyl ribonucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (15)..(15): LNA nucleotide
  misc_feature (16)..(19): 2′-O-methyl ribonucleotide
acuggtacaagggttggga

SEQ ID NO: 18 - DNA, 17 bp, Artificial Sequence
  oligonucleotide block_miR-126-3p_v3
  misc_feature (1)..(4): 2′-O-methyl ribonucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (14)..(17): 2′-O-methyl ribonucleotide
cgcattattactcacgg

SEQ ID NO: 19 - DNA, 21 bp, Artificial Sequence
  oligonucleotide block_let-7f_v3
  misc_feature (1)..(3): 2′-O-methyl ribonucleotide
  misc_feature (4)..(4): LNA nucleotide
  misc_feature (6)..(6): LNA nucleotide
  misc_feature (9)..(9): LNA nucleotide
  misc_feature (11)..(11): LNA nucleotide
  misc_feature (18)..(21): 2′-O-methyl ribonucleotide
aactatacaatctactaccuc

SEQ ID NO: 20 - DNA, 17 bp, Artificial Sequence
  oligonucleotide block_miR-423-5p_v3
  misc_feature (1)..(5): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (15)..(17): 2′-O-methyl ribonucleotide
agucucgctctctgccc

SEQ ID NO: 21 - DNA, 22 bp, Artificial Sequence
  oligonucleotide block_hY3_v3
  misc_feature (1)..(5): 2′-O-methyl ribonucleotide
  misc_feature (10)..(10): LNA nucleotide
  misc_feature (12)..(12): LNA nucleotide
  misc_feature (14)..(14): LNA nucleotide
  misc_feature (16)..(16): LNA nucleotide
  misc_feature (18)..(18): LNA nucleotide
  misc_feature (19)..(22): 2′-O-methyl ribonucleotide
cuagucaagtgaagcagtggga

SEQ ID NO: 22 - DNA, 18 bp, Artificial Sequence
  oligonucleotide block_miR-26a-5p_v3
  misc_feature (1)..(4): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (15)..(18): 2′-O-methyl ribonucleotide
ccuatcctggattacuug

SEQ ID NO: 23 - DNA, 18 bp, Artificial Sequence
  oligonucleotide block_miR-342-3p_v3
  misc_feature (1)..(4): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (15)..(18): 2′-O-methyl ribonucleotide
gggugcgatttctgugug

SEQ ID NO: 24 - DNA, 18 bp, Artificial Sequence
  oligonucleotide block_miR-128-3p_v3
  misc_feature (1)..(4): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (15)..(18): 2′-O-methyl ribonucleotide
agagaccggttcacugug

SEQ ID NO: 25 - DNA, 22 bp, Artificial Sequence
  oligonucleotide block_hY1_v3
  misc_feature (1)..(5): 2′-O-methyl ribonucleotide
  misc_feature (10)..(10): LNA nucleotide
  misc_feature (12)..(12): LNA nucleotide
  misc_feature (14)..(14): LNA nucleotide
  misc_feature (16)..(16): LNA nucleotide
  misc_feature (18)..(18): LNA nucleotide
  misc_feature (19)..(22): 2′-O-methyl ribonucleotide
cuagucaagtgcagtagtgaga

SEQ ID NO: 26 - DNA, 20 bp, Artificial Sequence
  oligonucleotide block_miR-425-5p_v3
  misc_feature (1)..(4): 2′-O-methyl ribonucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (16)..(16): LNA nucleotide
  misc_feature (17)..(20): 2′-O-methyl ribonucleotide
aacgggagtgatcgtgucau

SEQ ID NO: 27 - DNA, 20 bp, Artificial Sequence
  oligonucleotide block_miR-125b-5p_v3
  misc_feature (1)..(4): 2′-O-methyl ribonucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (16)..(16): LNA nucleotide
  misc_feature (17)..(20): 2′-O-methyl ribonucleotide
cacaagttagggtctcaggg

SEQ ID NO: 28 - DNA, 19 bp, Artificial Sequence
  oligonucleotide block_miR-4732-3p_v3
  misc_feature (1)..(4): 2′-O-methyl ribonucleotide
  misc_feature (7)..(7): LNA nucleotide
  misc_feature (9)..(9): LNA nucleotide
  misc_feature (11)..(11): LNA nucleotide
  misc_feature (13)..(13): LNA nucleotide
  misc_feature (15)..(15): LNA nucleotide
  misc_feature (16)..(19): 2′-O-methyl ribonucleotide
agaacaggacaggtcaggg

SEQ ID NO: 29 - DNA, 22 bp, Artificial Sequence
  oligonucleotide block_miR-30c-5p_v3
  misc_feature (1)..(4): 2′-O-methyl ribonucleotide
  misc_feature (9)..(9): LNA nucleotide
  misc_feature (11)..(11): LNA nucleotide
  misc_feature (13)..(13): LNA nucleotide
  misc_feature (16)..(16): LNA nucleotide
  misc_feature (18)..(18): LNA nucleotide
  misc_feature (19)..(22): 2′-O-methyl ribonucleotide
cugagagtgtaggatgttuaca

SEQ ID NO: 30 - DNA, 20 bp, Artificial Sequence
  oligonucleotide block_let-7g_v3
  misc_feature (1)..(4): 2′-O-methyl ribonucleotide
  misc_feature (5)..(5): LNA nucleotide
  misc_feature (7)..(7): LNA nucleotide
  misc_feature (11)..(11): LNA nucleotide
  misc_feature (13)..(13): LNA nucleotide
  misc_feature (15)..(15): LNA nucleotide
  misc_feature (17)..(20): 2′-O-methyl ribonucleotide
acugtacaaactactaccuc

SEQ ID NO: 31 - DNA, 20 bp, Artificial Sequence
  oligonucleotide block_miR-484_v3
  misc_feature (1)..(4): 2′-O-methyl ribonucleotide
  misc_feature (6)..(6): LNA nucleotide
  misc_feature (9)..(9): LNA nucleotide
  misc_feature (11)..(11): LNA nucleotide
  misc_feature (13)..(13): LNA nucleotide
  misc_feature (17)..(20): 2′-O-methyl ribonucleotide
ucgggaggggactgagccug

SEQ ID NO: 32 - DNA, 18 bp, Artificial Sequence
  oligonucleotide block_miR-92a-3p_v3b
  misc_feature (1)..(4): 2′-O-methyl ribonucleotide
  misc_feature (5)..(5): LNA nucleotide
  misc_feature (9)..(9): LNA nucleotide
  misc_feature (11)..(11): LNA nucleotide
  misc_feature (13)..(13): LNA nucleotide
  misc_feature (14)..(18): 2′-O-methyl ribonucleotide
acaggccgggacaagugc

SEQ ID NO: 33 - DNA, 18 bp, Artificial Sequence
  oligonucleotide block_miR-142-5p_v3b
  misc_feature (1)..(4): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (15)..(18): 2′-O-methyl ribonucleotide
agugctttctacttuaug

SEQ ID NO: 34 - RNA, 18 nt, Homo sapiens
guacugagcugccccgag

SEQ ID NO: 35 - RNA, 18 nt, Homo sapiens
gcagcacguaaauauugg

SEQ ID NO: 36 - RNA, 18 nt, Homo sapiens
cggaaucccaaaagcagc

SEQ ID NO: 37 - RNA, 20 nt, Homo sapiens
cccacugcuaaauuugacug

SEQ ID NO: 38 - RNA, 17 nt, Homo sapiens
gcacuugucucggucug

SEQ ID NO: 39 - RNA, 17 nt, Homo sapiens
caguuugucaaauaccc

SEQ ID NO: 40 - RNA, 21 nt, Homo sapiens
gagguaguagguuguauaguu

SEQ ID NO: 41 - RNA, 19 nt, Homo sapiens
gguaguaguuugugcuguu

SEQ ID NO: 42 - RNA, 18 nt, Homo sapiens
aagugcuguucgugcagg

SEQ ID NO: 43 - RNA, 17 nt, Homo sapiens
ccacaggguagaaccac

SEQ ID NO: 44 - RNA, 20 nt, Homo sapiens
uguaaacauccccgacugga

SEQ ID NO: 45 - RNA, 17 nt, Homo sapiens
ggcaaugguagaacuca

SEQ ID NO: 46 - RNA, 20 nt, Homo sapiens
ggagagaaaggcaguuccug

SEQ ID NO: 47 - RNA, 19 nt, Homo sapiens
agcauuguacagggcuaug

SEQ ID NO: 48 - RNA, 21 nt, Homo sapiens
gagguaguagguugugugguu

SEQ ID NO: 49 - RNA, 19 nt, Homo sapiens
accguuaccauuacugagu

SEQ ID NO: 50 - RNA, 19 nt, Homo sapiens
ucccaacccuuguaccagu

SEQ ID NO: 51 - RNA, 17 nt, Homo sapiens
ccgugaguaauaaugcg

SEQ ID NO: 52 - RNA, 21 nt, Homo sapiens
gagguaguagauuguauaguu

SEQ ID NO: 53 - RNA, 17 nt, Homo sapiens
gggcagagagcgagacu

SEQ ID NO: 54 - RNA, 22 nt, Homo sapiens
ucccacugcuucacuugacuag

SEQ ID NO: 55 - RNA, 18 nt, Homo sapiens
caaguaauccaggauagg

SEQ ID NO: 56 - RNA, 18 nt, Homo sapiens
cacacagaaaucgcaccc

SEQ ID NO: 57 - RNA, 18 nt, Homo sapiens
cacagugaaccggucucu

SEQ ID NO: 58 - RNA, 22 nt, Homo sapiens
ucucacuacugcacuugacuag

SEQ ID NO: 59 - RNA, 20 nt, Homo sapiens
augacacgaucacucccguu

SEQ ID NO: 60 - RNA, 20 nt, Homo sapiens
cccugagacccuaacuugug

SEQ ID NO: 61 - RNA, 19 nt, Homo sapiens
cccugaccuguccuguucu

SEQ ID NO: 62 - RNA, 22 nt, Homo sapiens
uguaaacauccuacacucucag

SEQ ID NO: 63 - RNA, 20 nt, Homo sapiens
gagguaguaguuuguacagu

SEQ ID NO: 64 - RNA, 20 nt, Homo sapiens
caggcucaguccccucccga

SEQ ID NO: 65 - RNA, 18 nt, Homo sapiens
gcacuugucccggccugu

SEQ ID NO: 66 - RNA, 18 nt, Homo sapiens
cauaaaguagaaagcacu

SEQ ID NO: 67 - RNA, 22 nt, Homo sapiens
uagcagcacguaaauauuggcg

SEQ ID NO: 68 - DNA, 18 bp, Artificial Sequence
  oligonucleotide block_miR-16-5p_v1
  misc_feature (2)..(2): LNA nucleotide
  misc_feature (5)..(5): LNA nucleotide
  misc_feature (7)..(7): LNA nucleotide
  misc_feature (9)..(9): LNA nucleotide
  misc_feature (11)..(11): LNA nucleotide
  misc_feature (14)..(14): LNA nucleotide
  misc_feature (17)..(17): LNA nucleotide
ccaatatttacgtgctgc

SEQ ID NO: 69 - DNA, 18 bp, Artificial Sequence
  oligonucleotide block_miR-16-5p_v2
  misc_feature (1)..(18): 2′-O-methyl ribonucleotide
ccaauauuuacgugcugc

SEQ ID NO: 70 - DNA, 19 bp, Artificial Sequence
  DNA linker sequence
aactgtaggcaccatcaat

SEQ ID NO: 71 - RNA, 26 nt, Artificial Sequence
  RNA adapter sequence
guucagaguucuacaguccgacgauc

SEQ ID NO: 72 - DNA, 52 bp, Artificial Sequence
  RT primer DNA oligonucleotide
  misc_feature (22)..(33): n is a, c, g, or t
  misc_feature (41)..(41): LNA nucleotide
  misc_feature (43)..(43): LNA nucleotide
agacgtgtgctcttccgatctnnnnnnnnnnnnattgatggtgcctacagtt

SEQ ID NO: 73 - DNA, 62 bp, Artificial Sequence
  forward primer
  misc_feature (30)..(37): 8 nucleotide index sequence
  misc_feature (30)..(37): n is a, c, g, or t
  misc_feature (61)..(62): linked by phosphorothioate bond
aatgatacggcgaccaccgagatctacacnnnnnnnnacacgttcagagttctacagtccga

SEQ ID NO: 74 - DNA, 66 bp, Artificial Sequence
  reverse primer
  misc_feature (27)..(34): 8 nucleotide index sequence
  misc_feature (27)..(34): n is a, c, g, or t
  misc_feature (65)..(66): linked by phosphorothioate bond
caagcagaagacggcatacgagatatnnnnnnnngactggagttcagacgtgtgctcttccgatct

SEQ ID NO: 75 - DNA, 30 bp, Artificial Sequence
  Custom index 2 primer
gatcgtcggactgtagaactctgaacgtgt

SEQ ID NO: 76 - DNA, 20 bp, Artificial Sequence
  oligonucleotide block_18S_1_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
cuggcaggatcaaccaggua

SEQ ID NO: 77 - DNA, 20 bp, Artificial Sequence
  oligonucleotide block_28S_2_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
cacgtctgatctgaggucgc

SEQ ID NO: 78 - DNA, 20 bp, Artificial Sequence
  oligonucleotide block_28S_9891_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
acuuccatggccaccguccu

SEQ ID NO: 79 - DNA, 22 bp, Artificial Sequence
  oligonucleotide block_rRNA_v3
  misc_feature (1)..(4): 2′-O-methyl ribonucleotide
  misc_feature (10)..(10): LNA nucleotide
  misc_feature (12)..(12): LNA nucleotide
  misc_feature (14)..(14): LNA nucleotide
  misc_feature (16)..(16): LNA nucleotide
  misc_feature (18)..(18): LNA nucleotide
  misc_feature (19)..(22): 2′-O-methyl ribonucleotide
aacccttgtgtcgagggcugac

SEQ ID NO: 80 - DNA, 20 bp, Artificial Sequence
  oligonucleotide block_5.8S_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
gugatccaccgctaagaguc

SEQ ID NO: 81 - DNA, 20 bp, Artificial Sequence
  oligonucleotide block_5S_v3a
  misc_feature (1)..(4): 2′-O-methyl ribonucleotide
  misc_feature (6)..(6): LNA nucleotide
  misc_feature (9)..(9): LNA nucleotide
  misc_feature (15)..(15): LNA nucleotide
  misc_feature (17)..(20): 2′-O-methyl ribonucleotide
agacgagatcgggcgcguuc

SEQ ID NO: 82 - DNA, 19 bp, Artificial Sequence
  oligonucleotide block_5S_v3b
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (16)..(19): 2′-O-methyl ribonucleotide
ggguggtatggccgtagac

SEQ ID NO: 83 - DNA, 20 bp, Artificial Sequence
  oligonucleotide block_5S_v3c
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
cccugcttagcttccgagau

SEQ ID NO: 84 - DNA, 20 bp, Artificial Sequence
  oligonucleotide block_5S_v3d
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
cccatccaagtactaaccag

SEQ ID NO: 85 - DNA, 20 bp, Artificial Sequence
  oligonucleotide block_hY1_v3a
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
agagtagaacaaggaguucg

SEQ ID NO: 86 - DNA, 20 bp, Artificial Sequence
  oligonucleotide block_hY1_v3b
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
acugtgaacaatcaatugag

SEQ ID NO: 87 - DNA, 20 bp, Artificial Sequence
  oligonucleotide block_hY1_v3c
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
ucactaccttcggaccagcc

SEQ ID NO: 88 - DNA, 20 bp, Artificial Sequence
  oligonucleotide block_hY3_v3a
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
ccactgcactcggaccagcc

SEQ ID NO: 89 - DNA, 18 bp, Artificial Sequence
  oligonucleotide block_hY4_v3
  misc_feature (1)..(4): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (14)..(14): LNA nucleotide
  misc_feature (15)..(18): 2′-O-methyl ribonucleotide
agucaaatttagcagugg

SEQ ID NO: 90 - DNA, 20 bp, Artificial Sequence
  oligonucleotide block_hY4_v3b
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
guugtataccaactttagug

SEQ ID NO: 91 - DNA, 20 bp, Artificial Sequence
  oligonucleotide block_hY4_v3c
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
cuaatgttaataagttcuga

SEQ ID NO: 92 - DNA, 20 bp, Artificial Sequence
  oligonucleotide block_hY4_v3d
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
ccactaccatcggaccagcc

SEQ ID NO: 93 - DNA, 20 bp, Artificial Sequence
  oligonucleotide block_hY5_v3a
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
uagucaagcgcggttguggg

SEQ ID NO: 94 - DNA, 20 bp, Artificial Sequence
  oligonucleotide block_hY5_v3b
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
ccacaacactcggaccaacu

SEQ ID NO: 95 - DNA, 20 bp, Artificial Sequence
  oligonucleotide block_snoRD12B_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
aaagctaagtcatcatauau

SEQ ID NO: 96 - DNA, 20 bp, Artificial Sequence
  oligonucleotide block_snorD18A_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
aaguggaatttcatcacuac

SEQ ID NO: 97 - DNA, 20 bp, Artificial Sequence
  oligonucleotide block_snoRD2_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
agaugattgccatcatuuca

SEQ ID NO: 98 - DNA, 20 bp, Artificial Sequence
  oligonucleotide block_snoRD26_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
uucgtaaaatcatccccgua

SEQ ID NO: 99 - DNA, 20 bp, Artificial Sequence
  oligonucleotide block_snorD27_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
auuutgtgttcatcatggag

SEQ ID NO: 100 - DNA, 20 bp, Artificial Sequence
  oligonucleotide block_snoRD29_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
uaguttgattcatcatagaa

SEQ ID NO: 101 - DNA, 20 bp, Artificial Sequence
  oligonucleotide block_snoRD30_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
ccaugtaagtcatcacaaac

SEQ ID NO: 102 - DNA, 20 bp, Artificial Sequence
  oligonucleotide block_snoRD38B_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
acaaagttttcatcacugag

SEQ ID NO: 103 - DNA, 20 bp, Artificial Sequence
  oligonucleotide block_snoRD42B_v3
  misc_feature (1)..(4): 2′-O-methyl ribonucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (15)..(15): LNA nucleotide
  misc_feature (17)..(20): 2′-O-methyl ribonucleotide
aaaacttttccatcataugc

SEQ ID NO: 104 - DNA, 20 bp, Artificial Sequence
  oligonucleotide block_snoRD43_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
ucaataagttcatcatcugu

SEQ ID NO: 105 - DNA, 19 bp, Artificial Sequence
  oligonucleotide block_snoRD44_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (16)..(19): 2′-O-methyl ribonucleotide
uuugcttatcatcatccag

SEQ ID NO: 106 - DNA, 20 bp, Artificial Sequence
  oligonucleotide block_snoRD49A_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
uuagtgatttcatcagagca

SEQ ID NO: 107 - DNA, 21 bp, Artificial Sequence
  oligonucleotide block_snoRD59B_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (18)..(21): 2′-O-methyl ribonucleotide
gucagaacgtactcatcagug

SEQ ID NO: 108 - DNA, 20 bp, Artificial Sequence
  oligonucleotide block_snoRD6_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
uuucgcccatcatcataaca

SEQ ID NO: 109 - DNA, 20 bp, Artificial Sequence
  oligonucleotide block_snoRD63_v3
  misc_feature (1)..(4): 2′-O-methyl ribonucleotide
  misc_feature (6)..(6): LNA nucleotide
  misc_feature (9)..(9): LNA nucleotide
  misc_feature (11)..(11): LNA nucleotide
  misc_feature (14)..(14): LNA nucleotide
  misc_feature (16)..(16): LNA nucleotide
  misc_feature (17)..(20): 2′-O-methyl ribonucleotide
gaauaaaatacatcatugca

SEQ ID NO: 110 - DNA, 20 bp, Artificial Sequence
  oligonucleotide block_snoRD69_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
auccagtttatcatcauuug

SEQ ID NO: 111 - DNA, 20 bp, Artificial Sequence
  oligonucleotide block_snoRD76_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
aauaaactgtcatcatugug

SEQ ID NO: 112 - DNA, 20 bp, Artificial Sequence
  oligonucleotide block_snoRD80_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
aacuatgttatcatcauugu

SEQ ID NO: 113 - DNA, 20 bp, Artificial Sequence
  oligonucleotide block_snoRD81_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
ggautgagatcatcatguau

SEQ ID NO: 114 - DNA, 16 bp, Artificial Sequence
  oligonucleotide block_tRNA_Ala_v3
  misc_feature (1)..(4): 2′-O-methyl ribonucleotide
  misc_feature (5)..(5): LNA nucleotide
  misc_feature (6)..(6): LNA nucleotide
  misc_feature (8)..(8): LNA nucleotide
  misc_feature (10)..(10): LNA nucleotide
  misc_feature (12)..(12): LNA nucleotide
  misc_feature (13)..(16): 2′-O-methyl ribonucleotide
ugguggagatgccggg

SEQ ID NO: 115 - DNA, 20 bp, Artificial Sequence
  oligonucleotide block_tRNAAla_v3a
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
cuaccatttgagctaauccc

SEQ ID NO: 116 - DNA, 20 bp, Artificial Sequence
  oligonucleotide block_tRNAArg_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
uauccattgcgccacggagc

SEQ ID NO: 117 - DNA, 20 bp, Artificial Sequence
  oligonucleotide block_tRNAGlu_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
uaaccactagaccaccaggg

SEQ ID NO: 118 - DNA, 20 bp, Artificial Sequence
  oligonucleotide block_tRNALeu_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
gaccgctcggccatccugac

SEQ ID NO: 119 - DNA, 20 bp, Artificial Sequence
  oligonucleotide block_tRNALys_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
cuaccgactgagctagccgg

SEQ ID NO: 120 - DNA, 20 bp, Artificial Sequence
  oligonucleotide block_tRNASeCys_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
accactgaggatcatccggg

SEQ ID NO: 121 - DNA, 20 bp, Artificial Sequence
  oligonucleotide block_tRNASer_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
aaccactcggccacgacuac

SEQ ID NO: 122 - DNA, 20 bp, Artificial Sequence
  oligonucleotide block_tRNAVal_v3
  misc_feature (1)..(4): 2′-O-methyl ribonucleotide
  misc_feature (6)..(6): LNA nucleotide
  misc_feature (9)..(9): LNA nucleotide
  misc_feature (12)..(12): LNA nucleotide
  misc_feature (15)..(15): LNA nucleotide
  misc_feature (17)..(20): 2′-O-methyl ribonucleotide
aaccactacactacggaaac

SEQ ID NO: 123 - RNA, 20 nt, Homo sapiens
uaccugguugauccugccag

SEQ ID NO: 124 - RNA, 20 nt, Homo sapiens
gcgaccucagaucagacgug

SEQ ID NO: 125 - RNA, 20 nt, Homo sapiens
aggacgguggccauggaagu

SEQ ID NO: 126 - RNA, 22 nt, Homo sapiens
gucagcccucgacacaaggguu

SEQ ID NO: 127 - RNA, 20 nt, Homo sapiens
gacucuuagcgguggaucac

SEQ ID NO: 128 - RNA, 20 nt, Homo sapiens
gaacgcgcccgaucucgucu

SEQ ID NO: 129 - RNA, 19 nt, Homo sapiens
gucuacggccauaccaccc

SEQ ID NO: 130 - RNA, 20 nt, Homo sapiens
aucucggaagcuaagcaggg

SEQ ID NO: 131 - RNA, 20 nt, Homo sapiens
cugguuaguacuuggauggg

SEQ ID NO: 132 - RNA, 20 nt, Homo sapiens
cgaacuccuuguucuacucu

SEQ ID NO: 133 - RNA, 20 nt, Homo sapiens
cucaauugauuguucacagu

SEQ ID NO: 134 - RNA, 20 nt, Homo sapiens
ggcugguccgaagguaguga

SEQ ID NO: 135 - RNA, 20 nt, Homo sapiens
ggcugguccgagugcagugg

SEQ ID NO: 136 - RNA, 18 nt, Homo sapiens
ccacugcuaaauuugacu

SEQ ID NO: 137 - RNA, 20 nt, Homo sapiens
cacuaaaguugguauacaac

SEQ ID NO: 138 - RNA, 20 nt, Homo sapiens
ucagaacuuauuaacauuag

SEQ ID NO: 139 - RNA, 20 nt, Homo sapiens
ggcugguccgaugguagugg

SEQ ID NO: 140 - RNA, 20 nt, Homo sapiens
cccacaaccgcgcuugacua

SEQ ID NO: 141 - RNA, 20 nt, Homo sapiens
aguugguccgaguguugugg

SEQ ID NO: 142 - RNA, 20 nt, Homo sapiens
auauaugaugacuuagcuuu

SEQ ID NO: 143 - RNA, 20 nt, Homo sapiens
guagugaugaaauuccacuu

SEQ ID NO: 144 - RNA, 20 nt, Homo sapiens
ugaaaugauggcaaucaucu

SEQ ID NO: 145 - RNA, 20 nt, Homo sapiens
uacggggaugauuuuacgaa

SEQ ID NO: 146 - RNA, 20 nt, Homo sapiens
cuccaugaugaacacaaaau

SEQ ID NO: 147 - RNA, 20 nt, Homo sapiens
uucuaugaugaaucaaacua

SEQ ID NO: 148 - RNA, 20 nt, Homo sapiens
guuugugaugacuuacaugg

SEQ ID NO: 149 - RNA, 20 nt, Homo sapiens
cucagugaugaaaacuuugu

SEQ ID NO: 150 - RNA, 20 nt, Homo sapiens
gcauaugauggaaaaguuuu

SEQ ID NO: 151 - RNA, 20 nt, Homo sapiens
acagaugaugaacuuauuga

SEQ ID NO: 152 - RNA, 19 nt, Homo sapiens
cuggaugaugauaagcaaa

SEQ ID NO: 153 - RNA, 20 nt, Homo sapiens
ugcucugaugaaaucacuaa

SEQ ID NO: 154 - RNA, 21 nt, Homo sapiens
cacugaugaguacguucugac

SEQ ID NO: 155 - RNA, 20 nt, Homo sapiens
uguuaugaugaugggcgaaa

SEQ ID NO: 156 - RNA, 20 nt, Homo sapiens
ugcaaugauguauuuuauuc

SEQ ID NO: 157 - RNA, 20 nt, Homo sapiens
caaaugaugauaaacuggau

SEQ ID NO: 158 - RNA, 20 nt, Homo sapiens
cacaaugaugacaguuuauu

SEQ ID NO: 159 - RNA, 20 nt, Homo sapiens
acaaugaugauaacauaguu

SEQ ID NO: 160 - RNA, 20 nt, Homo sapiens
auacaugaugaucucaaucc

SEQ ID NO: 161 - RNA, 16 nt, Homo sapiens
cccggcaucuccacca

SEQ ID NO: 162 - RNA, 20 nt, Homo sapiens
gggauuagcucaaaugguag

SEQ ID NO: 163 - RNA, 20 nt, Homo sapiens
gcuccguggcgcaauggaua

SEQ ID NO: 164 - RNA, 20 nt, Homo sapiens
cccugguggucuagugguua

SEQ ID NO: 165 - RNA, 20 nt, Homo sapiens
gucaggauggccgagcgguc

SEQ ID NO: 166 - RNA, 20 nt, Homo sapiens
ccggcuagcucagucgguag

SEQ ID NO: 167 - RNA, 20 nt, Homo sapiens
cccggaugauccucaguggu

SEQ ID NO: 168 - RNA, 20 nt, Homo sapiens
guagucguggccgagugguu

SEQ ID NO: 169 - RNA, 20 nt, Homo sapiens
guuuccguaguguagugguu

(19) United States
(12) Patent Application Publication
(10) Pub. No.: US 2025/0270552 A1
(43) Pub. Date: Aug. 28, 2025

(54) TARGETED INHIBITION OF REVERSE TRANSCRIPTION USING ANTISENSE OLIGOS

(71) Applicant: Hummingbird Dagnostics GmbH, Heidelberg (DE)

(72) Inventors: Rastislav Horos, Heidelberg (DE); Timothy Rajakumar, Heidelberg (DE); Bruno Steinkraus, Heidelberg (DE); Mustafa Kahraman, Saarbrucken (DE)

(21) Appl. No.: 18/702,985

(22) PCT Filed: Jun. 28, 2022
(86) PCT No.: PCT/EP2022/067813

§ 371 Date: Apr. 19, 2024

(30) Foreign Application Priority Data
Oct. 22, 2021 (EP) 21204208.9

(51) Int. Cl. C12N 15/113

(52) U.S. Cl. CPC C12N 15/113; C12N 2310/113; C12N 2310/321; C12N 2310/3231; C12N 2310/33

(57) ABSTRACT

The present invention relates to the targeted inhibition of reverse transcription using antisense oligonucleotides (blocking oligonucleotides). Antisense oligonucleotides (blocking oligonucleotides) and methods wherein said antisense oligonucleotides are used are specifically disclosed.

The present invention relates to the targeted inhibition of reverse transcription using antisense oligonucleotides (blocking oligonucleotides). Antisense oligonucleotides (blocking oligonucleotides) and methods wherein said antisense oligonucleotides are used are specifically disclosed.

### BACKGROUND OF THE INVENTION

Small non-coding RNAs, such as miRNAs, can be used due to their diversity and characteristic expression for the diagnosis of human diseases. In order to fully encompass the complexity of small non-coding RNAs in a biological sample e.g. isolated from human peripheral blood, and to use this information for the discovery of potential biomarkers, biochemical methods are employed that convert small non-coding RNAs into a cDNA library for sequencing such as high-throughput next-generation sequencing (NGS). This library preparation process is typically based on enzymatic reactions (ligations, reverse transcription, polymerization) and aims for unbiased and truthful representation of the small non-coding RNA complexity in the initial RNA sample.

Next generation sequencing (NGS) has enabled an unprecedented insight into biological function though the ability to sequence millions of molecules in parallel (reads). The number of reads corresponding to a given molecular species within a sample is correlated to its relative abundance. This is problematic in samples that contain a few highly abundant species to which the majority of reads are allocated and prevents the accurate detection and quantification of the multitude of less abundant species. Inclusion of said highly abundant species in sequencing consumes read depth and therethrough increases the costs of sequencing while producing irrelevant data.

Similarly, it has been observed that certain small RNA sequencing library preparation kits highly favor the biased incorporation of certain RNA species such that they too are read many times at the expense of less abundant or less efficiently incorporated species. This bias is caused by intermolecular pairing of kits components (e.g. adapters) and certain small RNAs, like miRNAs, which leads to formation of RNA double-stranded structures which are favorable substrates for ligation reactions.

Currently, some RNA NGS kits contain components that can inhibit the presence of highly abundant RNA species (e.g. rRNA) in the library pool. However, these kits are not so effective. In addition, said kits do not allow the inhibition of the presence of highly abundant miRNAs, miRNA isoforms (isomiRs), or Y-RNA molecules. In addition, said kits do not allow the inhibition of the presence of highly abundant small nucleolar ribonucleic acids (snoRNAs), ribosomal ribonucleic acid (rRNA) fragments, or transfer RNAs (tRNAs).

Thus, there is still a need for improved kits and methodologies.

To improve the current kits and methodologies, the present inventors have developed new blocking oligonucleotides and methods wherein said blocking oligonucleotides are used. In this way, any abundant small RNA of choice can be targeted and its reverse transcription can be inhibited/prevented which in turn significantly decreases its presence in the NGS datasets. Accordingly, less abundant small RNA molecules can accurately be detected and quantified in a fast, easy, and cost-effective way.

### SUMMARY OF THE INVENTION

In a first aspect, the present invention relates to a blocking oligonucleotide comprising one or more modified ribonucleotides and/or one or more locked nucleotides.

In a second aspect, the present invention relates to set comprising at least two blocking oligonucleotides according to the first aspect.

In a third aspect, the present invention relates to a method for inhibiting cDNA synthesis of one or more unwanted RNA molecules in an RNA sample during reverse transcription comprising the steps of:
(i) providing a mixture containing an RNA sample comprising one or more desired RNA molecules and one or more unwanted RNA molecules and one or more blocking oligonucleotides according to the first aspect (or blocking oligonucleotides comprised in the set according to the second aspect), wherein the one or more blocking oligonucleotides are annealed to the one or more unwanted RNA molecules, and
(ii) reverse transcribing the one or more desired RNA molecules, thereby obtaining one or more cDNA products from said desired RNA molecule(s) and inhibiting cDNA synthesis of the one or more unwanted RNA molecules.

In a fourth aspect, the present invention relates to a method for (improving) cDNA synthesis of one or more desired RNA molecules in an RNA sample during reverse transcription comprising the steps of:
(i) providing a mixture containing an RNA sample comprising one or more desired RNA molecules and one or more unwanted RNA molecules and one or more blocking oligonucleotides according to the first aspect (or blocking oligonucleotides comprised in the set according to the second aspect), wherein the one or more blocking oligonucleotides are annealed to the one or more unwanted RNA molecules, and
(ii) reverse transcribing the one or more desired RNA molecules, thereby obtaining one or more cDNA products from said desired RNA molecule(s) and inhibiting cDNA synthesis of the one or more unwanted RNA molecules.

In a fifth aspect, the present invention relates to a method for producing one or more double stranded cDNA products from one or more desired RNA molecules comprising the steps of:
(i) carrying out the method according to the third or fourth aspect, and
(ii) amplifying the one or more cDNA products, thereby producing one or more double stranded cDNA products from one or more desired RNA molecules.

In a sixth aspect, the present invention relates to a method for determining a profile of one or more desired RNA molecules comprising the step of:
sequencing the one or more double stranded cDNA products from the one or more desired RNA molecules produced by the method according to the fifth aspect.

In a seventh aspect, the present invention relates to the use of the blocking oligonucleotide according to the first aspect for inhibiting cDNA synthesis of an unwanted RNA molecule in an RNA sample during reverse transcription.

In an eighth aspect, the present invention relates to the use of the set of oligonucleotides according to the second for inhibiting cDNA synthesis of unwanted RNA molecules in an RNA sample during reverse transcription.

In a ninth aspect, the present invention relates to a kit comprising
the oligonucleotide according to the first aspect, or
the set comprising at least two blocking oligonucleotides according to the second aspect.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention. In the event of a conflict between the definitions or teachings of such incorporated references and definitions or teachings recited in the present specification, the text of the present specification takes precedence.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The term “nucleotide”, as used herein, refers to an organic molecule consisting of a nucleoside and a phosphate. In particular, a nucleotide is composed of three subunit molecules: a nucleobase, a five-carbon sugar (ribose or deoxyribose), and a phosphate group consisting of one to three phosphates. The four nucleobases in DNA are guanine, adenine, cytosine and thymine; in RNA, uracil is used in place of thymine. The nucleotide serves as monomeric unit of nucleic acid polymers, such as deoxyribonucleotide acid (DNA) or ribonucleotide acid (RNA). Thus, the nucleotide is a molecular building-block of DNA and RNA.

The term “nucleoside”, as used herein, refers to a glycosylamine that can be thought of as nucleotide without a phosphate group. A nucleoside consists simply of a nucleobase (also termed a nitrogenous base) and a five-carbon sugar (ribose or 2′-deoxyribose) whereas a nucleotide is composed of a nucleobase, a five-carbon sugar, and one or more phosphate groups. In a nucleoside, the anomeric carbon is linked through a glycosidic bond to the N9 of a purine or the N1 of a pyrimindine.

The term “nucleotide sequence”, as used herein, refers to a stretch of single-stranded or double-stranded nucleotide monomers, including without limitation, 2′-deoxyribonucleotides (DNA) and ribonucleotides (RNA) linked by internucleotide phosphodiester bond linkages, or internucleotide analogs, and associated counter ions, e.g. H+, NH4+, trialkylammonium, Mg2+, Na+, and the like. A nucleotide sequence may be composed entirely of deoxyribonucleotides, entirely of ribonucleotides, or chimeric mixtures thereof and may include nucleotide analogs. The term “polynucleotide”, as used herein, preferably refers to a nucleotide sequence having a length of at least 23 nucleotides. The term “oligonucleotide”, as used herein, preferably refers to a nucleotide sequence having a length of between 2 and 22 nucleotides. The blocking oligonucleotide of the present invention preferably encompasses between 16 and 22, e.g. 16, 17, 18, 19, 20, 21, or 22, nucleotides. A polynucleotide or oligonucleotide may be composed entirely of deoxyribonucleotides, entirely of ribonucleotides, or chimeric mixtures thereof and may include nucleotide analogs.

The nucleotide monomer units may comprise any of the nucleotides described herein, including, but not limited to, nucleotides and/or nucleotide analogs.

The term “nucleotide analog”, as used herein, includes synthetic analogs having modified base moieties, modified sugar moieties, and/or modified phosphate ester moieties. Phosphate analogs generally comprise analogs of phosphate wherein the phosphorous atom is in the +5 oxidation state and one or more of the oxygen atoms is replaced with a non-oxygen moiety, e.g. sulfur. Exemplary phosphate analogs include: phosphorothioate, phosphorodithioate, phosphoroselenoate, phosphorodiselenoate, phosphoroanilothioate, phosphoranilidate, phosphoramidate, boronophosphates, including associated counterions, e.g. H+, NH4+, Na+. Exemplary base analogs include: 2,6-diaminopurine, hypoxanthine, pseudouridine, C-5-propyne, isocytosine, isoguanine, 2-thiopyrimidine. Exemplary sugar analogs include: 2′- or 3′-modifications where the 2′- or 3′-position is hydrogen, hydroxy, alkoxy, e.g., methoxy, ethoxy, allyloxy, isopropoxy, butoxy, isobutoxy and phenoxy, azido, amino or alkylamino, fluoro, chloro, and bromo.

In one embodiment, one or more modified ribonucleotides are present in/part of the blocking oligonucleotides of the present invention. Particularly, the one or more modified ribonucleotides are 2′-o-methyl ribonucleotides.

The term “blocking oligonucleotide”, as used herein, refers to an oligonucleotide that is reverse complementary to at least a region of an unwanted RNA molecule. In one particular embodiment, the oligonucleotide is reverse complementary to the entire length of the (core structure of the) unwanted RNA molecule.

The term “core sequence”, as used herein, refers to a region that is all RNA molecules in common, e.g. an unwanted miRNA molecule and all isomiR molecules belonging to this unwanted miRNA molecule, whereas these said isomiRs typically, but not exclusively, differ from the unwanted miRNA sequence by nucleotide sequence changes on the extreme 5′ position or 3′ position, or both. The terms “core sequence” or “core region” are interchangeably used herein.

The term “blocking oligonucleotide”, as used herein, further refers to an oligonucleotide that is capable of stably binding to a region of an unwanted RNA molecule. The blocking oligonucleotide may be described as “targeting” the region of the unwanted RNA molecule. Due to this “targeting”, the blocking oligonucleotide is able to inhibit cDNA synthesis using the (core sequence) region of the unwanted RNA molecule. The formed duplex between the unwanted RNA molecule and the blocking oligonucleotide makes a reverse transcription reaction inefficient as the reverse transcriptase requires a single stranded RNA molecule as template.

An oligonucleotide is capable of stably binding to a region of an RNA molecule if the oligonucleotide anneals to the region of the RNA molecule and stays bound to the region of the RNA molecule during reverse transcription of an RNA sample comprising the RNA molecule. The blocking oligonucleotide of the present invention contains one or more modified ribonucleotides and/or one or more locked nucleotides that increase the binding between the oligonucleotide and the (core) region of the unwanted RNA molecule compared to an oligonucleotide with the same sequence but without one or more modified ribonucleotides and/or one or more locked nucleotides.

The one or more modified ribonucleotides and/or the one or more locked nucleotides comprised in the blocking oligonucleotide increase the binding between the blocking oligonucleotide and a (core) region of an unwanted RNA molecule compared to an oligonucleotide with the same sequence but without the one or more modified ribonucleotides and/or one or more locked nucleotides if they increase the melting temperature (Tm) of the duplex formed between the blocking oligonucleotide comprising the one or more modified ribonucleotides and/or the one or more locked nucleotides and the region of the unwanted RNA molecule compared to the melting temperature (Tm) of the duplex formed between the oligonucleotide with the same sequence but without the one or more modified ribonucleotides and/or the one or more locked nucleotides and the (core) region of the unwanted RNA molecule measured under the same conditions.

The melting temperature (Tm) of an oligonucleotide as described herein is the temperature at which 50% of the oligonucleotide is duplexed with its perfect complement and 50% is free. The Tm is determined by measuring the absorbance change of the oligonucleotide with its complement as a function of temperature (i.e. generating a melting curve). The Tm is the reading halfway between the double-stranded DNA and single-stranded DNA plateaus in the melting curve. The Tm of duplexes formed between the blocking oligonucleotides as described herein and regions of unwanted RNA molecules to which the blocking oligonucleotides are reverse complementary range from between 4° and 85° C., preferably from between 6° and 75° C., as predicted by the freely available online tools designed to estimate such Tm.

The (core) region of the unwanted RNA species to which the blocking oligonucleotide is reverse complementary may be at least 10 nucleotides in length, such as at least 11, 12, 13, 14, 15, 16, 17, 18, 19, 20, 21 nucleotides in length or 22 nucleotides in length. Such a region may be at most 30 nucleotides in length, such as at most 30, 29, 28, 27, 26, 25, 24, 23, or 22 nucleotides in length. In one preferred embodiment, the blocking oligonucleotide is reverse complementary to a (core) region of between 14 and 20 nucleotides in length, e.g. 14, 15, 16, 17, 18, 19, 20, 21, or 22 nucleotides in length, of an unwanted RNA molecule. In one more preferred embodiment, the blocking oligonucleotide is reverse complementary to a (core) region of between 16 and 22 nucleotides in length, e.g. 16, 17, 18, 19, 20, 21, or 22 nucleotides in length, of an unwanted RNA molecule.

The term “locked nucleotides (LNAs)”, as used herein, refers to modified nucleotides, e.g. deoxyribonucleotides or ribonucleotides, in which the 2′-O and 4′-C atoms of the ribose are joined through a methylene bridge. This additional bridge limits the flexibility normally associated with the ring, essentially locking the structure into a rigid conformation. These nucleic acid analogs are also referred to in some circles as “inaccessible nucleotides”. LNA nucleotides can be mixed with DNA or RNA residues in the polynucleotide/oligonucleotide in effect hybridizing with DNA or RNA according to Watson-Crick base-pairing rules. The inflexible nature of these molecules greatly enhances hybridization stability. Specifically, the locked ribose conformation enhances base stacking and backbone pre-organization, which significantly increases the hybridization properties (melting temperature) of oligonucleotides. Further, polynucleotides/oligonucleotides containing LNAs offer tremendous discriminatory power, allowing these molecules to distinguish between exact match and mismatched complementary target sequences with very little difficulty. An increase in the duplex melting temperature can be between 2 and 8° C., preferably between 4 and 6° C., per LNA nucleotide when incorporated into the blocking oligonucleotide as described herein. DNA or RNA oligonucleotides that comprise one or more LNA nucleotides can also be designated as “LNA oligonucleotides”.

In one embodiment, one or more locked nucleotides are present in/part of the blocking oligonucleotides of the present invention. Particularly, the one or more locked nucleotides are LNA-ribonucleotides or LNA-deoxyribonucleotides. More particularly, the one or more locked nucleotides are selected from the group consisting of LNA-adenine, LNA-guanine, LNA-cytosine, and LNA-thymine.

In one alternative embodiment, one or more modified ribonucleotides are present in/part of the blocking oligonucleotides of the present invention. Particularly, the one or more modified ribonucleotides are 2′-o-methyl ribonucleotides.

In one preferred embodiment, one or more modified ribonucleotides and/or one or more locked nucleotides are present in/part of the blocking oligonucleotides of the present invention. Particularly, the one or more modified ribonucleotides are 2′-o-methyl ribonucleotides and/or the one or more locked nucleotides are LNA-ribonucleotides or LNA-deoxyribonucleotides. More particularly, the one or more locked nucleotides are selected from the group consisting of LNA-adenine, LNA-guanine, LNA-cytosine, and LNA-thymine.

The term “modified ribonucleotides”, as used herein, refers to naturally occurring or synthetized chemical modifications of ribonucleotides. In one preferred embodiment, the modified ribonucleotides are 2′-o-methyl ribonucleotide(s). An increase in the duplex melting temperature of between 1 and 8° C., preferably of between 3 and 5° C., can be reached per 2′-o-methyl ribonucleotide when incorporated into the blocking oligonucleotide.

The term “unwanted RNA molecules”, as used herein, refers to RNA molecules undesired in an initial RNA composition or sample for a given downstream manipulation or analysis of the RNA composition or sample. Such RNA molecules are not the targets of, but may interfere with, downstream manipulation or analysis. The unwanted RNA molecules may be any undesired RNA molecules present in the initial RNA composition or sample. The unwanted RNA molecules may have or comprise any sequence as long as they are distinguishable by their sequence from the remaining RNA population of interest (i.e. desired/wanted RNA molecules) to allow a sequence-specific design of blocking oligonucleotides.

As mentioned above, next generation sequencing (NGS) has enabled an unprecedented insight into biological function though the ability to sequence millions of molecules in parallel (reads). The number of reads corresponding to a given molecular species within a sample is correlated to its relative abundance. This is problematic in samples that contain a few highly abundant species to which the majority of reads are allocated and prevents the accurate detection and quantification of the multitude of less abundant species. Inclusion of said highly abundant species in sequencing consumes read depth and therethrough increases the costs of sequencing while producing irrelevant data.

Thus, in one preferred embodiment, the unwanted RNA molecules are abundant RNA molecules. The term “abundant RNA molecules”, as used herein, refers to RNA entities that consume more than 0.05% of total processed reads in a small RNA NGS experiment. The skilled person is able to determine the abundance of RNA molecules, e.g. in an NGS experiment.

In one more preferred embodiment, the unwanted RNA molecules are abundant non-coding RNA molecules having a length of <200 ribonucleotides.

In one even more preferred embodiment, the unwanted RNA molecules are abundant miRNAs, miRNA isoforms (isomiRs), Y-RNAs, or Y-RNA fragments, that are not full-length Y-RNAs. Alternatively, the unwanted RNA molecules are abundant small nucleolar ribonucleic acids (snoRNAs), ribosomal ribonucleic acid (rRNA) fragments, that are not full-length rRNAs, or transfer RNAs (tRNAs). Specifically, the unwanted RNA molecules are abundant miRNAs, miRNA isoforms (isomiRs), Y-RNAs, Y-RNA fragments, that are not full-length Y-RNAs, snoRNAs, rRNA fragments that are not full length rRNAs, and/or tRNAs.

The terms “abundant miRNAs” or “abundant miRNA isoforms (isomiRs)”, as used herein, refer to miRNA or miRNA isoform (isomiR) entities that consume more than 0.05% of total processed reads in a small RNA NGS experiment. The skilled person is able to determine the abundance of miRNAs or miRNA isoforms (isomiRs), e.g. in an NGS experiment.

The term “abundant Y-RNAs or fragments thereof”, as used herein, refers to Y-RNA or Y-RNA fragment entities that consume more than 0.05% of total processed reads from a small RNA NGS experiment. The skilled person is able to determine the abundance of Y-RNAs or fragments thereof, e.g. in an NGS experiment.

The term “abundant snoRNAs”, as used herein, refers to snoRNA entities that consume more than 0.05% of total processed reads from a small RNA NGS experiment. The skilled person is able to determine the abundance of snoRNAs, e.g. in an NGS experiment.

The term “abundant rRNA fragments”, as used herein, refers to rRNA entities that consume more than 0.05% of total processed reads from a small RNA NGS experiment. The skilled person is able to determine the abundance of rRNA fragments, e.g. in an NGS experiment.

The term “abundant tRNAs”, as used herein, refers to tRNA entities that consume more than 0.05% of total processed reads from a small RNA NGS experiment. The skilled person is able to determine the abundance of tRNAs, e.g. in an NGS experiment.

In one most preferred embodiment, the unwanted RNA molecules are abundant miRNAs and/or miRNA isoforms (isomiRs).

The present inventors have developed new blocking oligonucleotides and methods wherein said blocking oligonucleotides are used. In this way, any abundant small RNA of choice can be targeted and its reverse transcription can be inhibited/prevented which in turn significantly decreases its presence in the NGS datasets. Accordingly, less abundant small RNA species can accurately be detected and quantified in a fast, easy, and cost-effective way.

The term “wanted RNA molecules”, as used herein, refers to RNA molecules desired in an initial RNA composition or sample for a given downstream manipulation or analysis of the RNA composition or sample. In addition to unwanted RNA molecules, an RNA sample also contains one or more desired RNA molecules. Desired RNA molecules can be any RNA molecules characteristic(s) of which (e.g. expression level or sequence) are of interest. In certain embodiments, the desired RNA molecules comprise RNA molecules those of which expression level changes (compared with a reference expression level) or sequence changes (compared with wild type sequences) are associated with a disease or disorder or with responsiveness to a treatment of a disease or disorder.

As mentioned above, next generation sequencing (NGS) has enabled an unprecedented insight into biological function though the ability to sequence millions of molecules in parallel (reads). The number of reads corresponding to a given molecular species within a sample is correlated to its relative abundance. This is problematic in samples that contain a few highly abundant species to which the majority of reads are allocated and prevents the accurate detection and quantification of the multitude of less abundant species.

Thus, in one preferred embodiment, the wanted RNA molecules are less abundant RNA molecules. The term “less abundant RNA molecules”, as used herein, refers to RNA entities that consume equal to or less than 0.05% of total processed reads in a small RNA NGS experiment. The skilled person is able to determine the abundance of RNA molecules, e.g. in an NGS experiment.

In one more preferred embodiment, the wanted RNA molecules are less abundant non-coding RNA molecules having a length of <200 ribonucleotides.

In one even more preferred embodiment, the wanted RNA molecules are less abundant miRNAs, miRNA isoforms (isomiRs), Y-RNAs, or Y-RNA fragments, that are not full-length Y-RNAs.

Alternatively, the wanted RNA molecules are less abundant small nucleolar ribonucleic acids (snoRNAs), ribosomal ribonucleic acid (rRNA) fragments, that are not full-length rRNAs, or transfer RNAs (tRNAs). Specifically, the wanted RNA molecules are less abundant miRNAs, miRNA isoforms (isomiRs), Y-RNAs, Y-RNA fragments, that are not full-length Y-RNAs, snoRNAs, rRNA fragments, that are not full-length rRNAs, and/or tRNAs.

The terms “less abundant miRNAs” or “less abundant miRNA isoforms (isomiRs)”, as used herein, refer to miRNA or miRNA isoform (isomiR) entities that consume equal to or less than 0.05% of total processed reads in a small RNA NGS experiment. The skilled person is able to determine the abundance of miRNAs or miRNA isoforms (isomiRs), e.g. in an NGS experiment.

The term “less abundant Y-RNAs or fragments thereof”, as used herein, refers to Y-RNAs or Y-RNA fragment entities that consume equal to or less than 0.05% of total processed reads from a small RNA NGS experiment. The skilled person is able to determine the abundance of Y-RNAs or fragments thereof, e.g. in an NGS experiment.

The term “less abundant snoRNAs”, as used herein, refers to snoRNA entities that consume equal to or less than 0.05% of total processed reads from a small RNA NGS experiment. The skilled person is able to determine the abundance of snoRNAs, e.g. in an NGS experiment.

The term “less abundant rRNA fragments”, as used herein, refers to rRNA fragment entities that consume equal to or less than 0.05% of total processed reads from a small RNA NGS experiment. The skilled person is able to determine the abundance of rRNA fragments, e.g. in an NGS experiment.

The term “less abundant tRNAs”, as used herein, refers to tRNA entities that consume equal to or less than 0.05% of total processed reads from a small RNA NGS experiment. The skilled person is able to determine the abundance of tRNAs, e.g. in an NGS experiment.

In one still more preferred embodiment, the wanted RNA molecules are a less abundant miRNAs and/or miRNA isoforms (isomiRs).

The present inventors have developed new blocking oligonucleotides and methods wherein said blocking oligonucleotides are used. In this way, any abundant small RNA of choice can be targeted and its reverse transcription can be inhibited/prevented which in turn significantly decreases its presence in the NGS datasets. Accordingly, less abundant small RNA species can accurately be detected and quantified in a fast, easy, and cost-effective way.

In one most preferred embodiment, the abundant RNA molecules are miRNAs and/or miRNA isoforms (isomiRs) and the less abundant RNA molecules are miRNAs and/or miRNA isoforms (isomiRs), wherein the abundant miRNAs or miRNA isoforms (isomiRs) and the less abundant miRNAs or miRNA isoforms (isomiRs) differ from each other/are different types.

The term “RNA sample”, as used herein, refers to an RNA-containing sample. Specifically, the RNA sample can be a starting material such as a biological sample. Alternatively, the RNA sample can be a sample containing RNAs isolated from a starting material such as a biological sample. An RNA sample may further contain DNAs isolated from the starting material. In other cases, an RNA sample is derived from a directly lysed sample without specific nucleic acid isolation. The starting material from which the RNA sample is generated can be any material that comprises RNA molecules. The starting material can be a biological sample or material, such as a cell sample, an environmental sample, a sample obtained from a body, in particular a body fluid sample, and a human, animal or plant tissue sample. Specific examples of biological samples include, but are not limited to, whole blood, blood fractions such as plasma, serum, or blood cells (e.g. erythrocytes, leukocytes and/or thrombocytes), urine, sputum, saliva, semen, lymphatic fluid, amniotic fluid, cerebrospinal fluid, peritoneal effusions, pleural effusions, fluid from cysts, synovial fluid, vitreous humor, aqueous humor, bursa fluid, eye washes, eye aspirates, pulmonary lavage, bone marrow aspirates, lung aspirates, biopsy samples, swab samples, animal (including human) or plant tissues, including but not limited to samples from liver, spleen, kidney, lung, intestine, brain, heart, muscle, pancreas, cell cultures, as well as lysates, extracts, or materials and fractions obtained from the samples described above or any cells and microorganisms and viruses that may be present on or in a sample and the like.

Materials obtained from clinical or forensic settings that contain RNA are also within the intended meaning of a starting material.

Preferably, the biological sample is a blood sample. More preferably, the blood sample is whole blood or a blood fraction such as plasma, serum, or blood cells (e.g. erythrocytes, leukocytes and/or thrombocytes). Particularly, the above biological samples encompass miRNAs, miRNA isoforms (isomiR), Y-RNAs, and/or Y-RNAs fragments. Alternatively, the above biological samples encompass snoRNAs, rRNA fragments, and/or tRNAs. More particularly, the above biological samples encompass miRNAs, miRNA isoforms (isomiR), Y-RNAs, Y-RNAs fragments, snoRNAs, rRNA fragments, and/or tRNAs.

The whole blood sample may be collected by means of a blood collection tube. It is, for example, collected in a PAXgene Blood RNA tube, in a Tempus Blood RNA tube, in an EDTA-tube, in a Na-citrate tube, Heparin-tube, or in an ACD-tube (Acid citrate dextrose).

The whole blood sample may also be collected by means of a bloodspot technique, e.g. using a Mitra Microsampling Device. This technique requires smaller sample volumes, typically 45-60 μl for humans or less. For example, the whole blood may be extracted from the patient via a finger prick with a needle or lancet. Thus, the whole blood sample may have the form of a blood drop. Said blood drop is then placed on an absorbent probe, e.g. a hydrophilic polymeric material such as cellulose, which is capable of absorbing the whole blood. Once sampling is complete, the blood spot is dried in air before transferring or mailing to labs for processing. Because the blood is dried, it is not considered hazardous. Thus, no special precautions need be taken in handling or shipping. Once at the analysis site, the desired components, e.g. miRNAs, are extracted from the dried blood spots into a supernatant which is then further analyzed. Nucleic acids can be isolated from a starting material according to methods known in the art to provide an RNA sample. The RNA sample may contain both DNA and RNA. In certain embodiments, the RNA sample contains predominantly RNA as DNA in the starting material has been removed or degraded. RNA in an RNA sample may be total RNA isolated from a starting material. Alternatively, RNA in an RNA sample may be a fraction of total RNA (e.g. a fraction encompassing miRNAs or isomiRs) isolated from a starting material where certain RNA molecules have been depleted or removed. In certain embodiments, an RNA sample is biological sample where specific nucleic acid isolation or separation has not been performed. Particularly, the RNA sample encompasses miRNAs and/or miRNA isoforms (isomiR) as wanted/unwanted RNA molecules. Said miRNAs and/or miRNA isoforms (isomiR) may be present as single stranded RNA molecules. Said miRNAs and/or miRNA isoforms (isomiR) may also be present attached to/linked to/flanked by adapter molecules after biochemical reactions. Especially, the adapter molecules are ligated to the 5′end of the miRNAs or miRNA isoforms (isomiR) (5′adapter) and to the 3′end of the miRNAs or miRNA isoforms (isomiR) (3′adapter). In other words, the miRNAs and/or miRNA isoforms (isomiR) as wanted/unwanted RNA molecules are flanked by 5′adapter and 3′adapter molecules. The adapters, at the 5′ and the 3′end of the miRNAs and/or miRNA isoforms (isomiR), enable the formation of ligated RNA molecules that can be used as templates for cDNA synthesis. In this way, not only miRNAs but also their variants, i.e. isomiRs, can be detected, which improves different molecular techniques such as next generation sequencing (NGS), cDNA library constructions, and diverse diagnostic methods.

As mentioned above, cDNA synthesis of unwanted RNA molecule is inhibited with the blocking oligonucleotides of the present invention which anneal to the unwanted RNA molecules and thereby prevent reverse transcription.

The “inhibition of cDNA synthesis of an unwanted RNA molecule” means that the amount of single stranded or double stranded cDNA generated using the unwanted RNA molecule as a template during reverse transcription is reduced at a statistically significant degree under a modified condition (e.g. in the presence of a blocking oligonucleotide comprising one or more modified ribonucleotides and/or one or more locked nucleotides) compared to the amount of single stranded or double stranded cDNA generated during reverse transcription under a reference condition (e.g. in the absence of a blocking oligonucleotide comprising one or more modified ribonucleotides and/or one or more locked nucleotides). The reduction in the amount of synthesized cDNA may be measured using qPCR or transcriptome sequencing as disclosed herein. Particularly, the cDNA synthesis of an unwanted RNA molecule is in inhibited to at least 20%, 30%, 40%, 50%, 60%, 70%, 80%, 90%, 95%, 99%, or even 100%.

In other words, the principle of the inhibition of cDNA synthesis of an unwanted RNA molecule is to form an oligo-target RNA double stranded structure with a high melting temperature that inhibits, slowdowns, or halts, reverse transcriptase processivity.

The inhibition of cDNA synthesis of an unwanted RNA molecule may be referred to as depletion of the unwanted RNA molecule. Even though the unwanted RNA molecule is not physically removed from an initial RNA sample, the involvement of the unwanted RNA molecule in the downstream manipulation or analysis of the initial RNA sample is reduced or eliminated due to the inhibition of cDNA synthesis of the unwanted RNA molecule.

The term “miRNA” (the designation “microRNA” is also possible), as used herein, refers to a single-stranded RNA molecule. The miRNA may be a molecule of 10 to 50 nucleotides in length, e.g. 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, not including optionally labels and/or elongated sequences (e.g. biotin stretches).

The miRNAs regulate gene expression and are encoded by genes from whose DNA they are transcribed but miRNAs are not translated into protein (i.e. miRNAs are non-coding RNAs). The genes encoding miRNAs are longer than the processed mature miRNA molecules. The miRNA is initially transcribed as a longer precursor molecule (>1000 nucleotides long) called a primary miRNA transcript (pri-miRNA). Pri-miRNAs have hairpin structures that are processed by the Drosha enzyme (as part of the microprocessor complex). After Drosha processing, the pri-miRNAs are only 60-100 nucleotides long, and are called precursor miRNAs (pre-miRNAs). At this point, the pre-miRNA is exported to the cytoplasm, where it encounters the Dicer enzyme. Dicer cuts the miRNA in two, resulting in duplexed miRNA strands. Traditionally, only one of these miRNA arms was considered important in gene regulation: the arm that is destined to be loaded into the RNA-induced silencing complex (RISC), and occurs at a higher concentration in the cell. This is often called the “guide” strand and is designated as miR. The other arm is called the “minor miRNA” or “passenger miRNA”, and is often designated as miR*. It was thought that passenger miRNAs were completely degraded, but deep sequencing studies have found that some minor miRNAs persist and in fact have a functional role in gene regulation. Due to these developments, the naming convention has shifted. Instead of the miR/miR* name scheme, a miR-5p/miR-3p nomenclature has been adopted. By the new system, the 5′ arm of the miRNA is always designated miR-5p and the 3′ arm is miR-3p. The present nomenclature is as follows: The prefix “miR” is followed by a dash and a number, the latter often indicating order of naming. For example, hsa-miR-16 was named and likely discovered prior to hsa-miR-342. A capitalized “miR-” refers to the mature forms of the miRNA (e.g. hsa-miR-16-5p and hsa-miR-16-3p), while the uncapitalized “mir-” refers to the pre-miRNA and the pri-miRNA (e.g. hsa-mir-16), and “MIR” refers to the gene that encodes them. However, as this is a recent change, literature will often refer to the original miR/miR* names. After processing, the duplexed miRNA strands are loaded onto an Argonaute (AGO) protein to form a precursor to the RISC. The complex causes the duplex to unwind, and the passenger RNA strand is discarded, leaving behind a mature RISC carrying the mature, single stranded miRNA. The miRNA remains part of the RISC as it silences the expression of its target genes. While this is the canonical pathway for miRNA biogenesis, a variety of others have been discovered. These include Drosha-independent pathways (such as the mirtron pathway, snoRNA-derived pathway, and shRNA-derived pathway) and Dicer-independent pathways (such as one that relies on AGO for cleavage, and another which is dependent on tRNaseZ).

The term “miRBase”, as used herein, refers to a well-established repository of validated miRNAs. The miRBase (www.mirbase.org) is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download. In October 2018, miRbase version 22.1 was released. This is the current version.

The term “isomiR” (or “miRNA isoform”), as used herein, refers to a miRNA that varies slightly in sequence, which results from variations in the cleavage site during miRNA biogenesis. In particular, imprecise cleavage of Drosha and Dicer or the turnover of miRNAs can result in miRNAs that are heterogeneous in length and/or sequence. IsomiRs (miRNA isoforms) can be divided into three main categories: 3′ isomiRs (trimmed or addition of one or more nucleotides at the 3′ position), 5′ isomiRs (trimmed or addition of one or more nucleotides at the 5′ position), and polymorphic isomiRs (some nucleotides within the sequence are different from the wild type mature miRNA sequence). It could be envisioned that the increased expression of miRNA variants, or individual isomiRs, lead to the loss or weakening of the function of the corresponding wild-type mature miRNA or result in the regulation of a different transcriptome. Recent studies suggest that isomiRs probably play vital roles in a variety of cancers, tissues, and cell types. The detection of miRNAs as well as isomiRs is, thus, absolutely required to accurately reflect the underlying biological situation and to make the right diagnostic and treatment decisions.

The blocking oligonucleotides designed by the present inventors allow the detection of unwanted miRNAs. Said blocking oligonucleotides designed by the present inventors also allow the detection of all isomiRs of said miRNAs. This is possible as the blocking oligonucleotides designed by the present inventors are reverse complementary to the core sequence of the miRNA as well as to all isomiRs of said miRNAs.

The term “Y-RNA molecule”, as used herein, refers to a non-coding RNA molecule. It is a component of the Ro60 ribonucleoprotein particle. Said molecule is, for example, necessary for DNA replication through interactions with chromatin and initiation proteins. Currently, 4 mature Y-RNAs are described to be expressed in human cells (hY1, hY3, hY4, hY5).

The term “snoRNA molecule”, as used herein, refers to a non-coding RNA molecule. It primarily guides chemical modifications of other RNAs, mainly ribosomal RNAs (rRNAs) or transfer RNAs (tRNAs). There are to classes of snoRNA, the C/D box snoRNAs, which are associated with methylation, and the H/ACA box snoRNAs, which are associated with pseudouridylation.

The term “rRNA fragment”, as used herein, refers to a non-coding RNA molecule. It is part of full-length ribosomal RNA that represents the major molecular building block of a ribosome. Thus, the rRNA fragment may also be designated as rRNA-derived fragment. While ribosomal RNAs can be between 120 to 4000 nucleotides long, the rRNA fragments referred to in this document are typically between 18 to 39 nucleotides long.

The term “tRNA molecule”, as used herein, refers to a non-coding RNA molecule. It is an adapter molecule composed of RNA, typically 76 to 90 nucleotides in length (in eukaryotes), that serves as physical link between the mRNA and the amino acid sequence of proteins. Transfer RNA (tRNA) does this by carrying an amino acid to the protein synthesizing machinery of a cell called the ribosome.

The term “annealing”, as used herein, refers to a process of heating and cooling down two single-stranded nucleotide sequence with reverse complementary sequences. Heat breaks all hydrogen bonds and cooling down allows new bonds to form between the sequences. During this process, the blocking oligonucleotides of the present invention attach to the unwanted RNA molecules and form a double stranded/hybrid structure.

Further disclosed herein is a method for producing a cDNA product from the wanted/desired RNA molecule comprised in the RNA sample. This method requires an extension reaction. As used herein, the term “extension reaction” refers to an elongation reaction in which the wanted/desired RNA molecule is extended via an RT primer, in particular in 5′ to 3′ direction, to form an “extension reaction product” comprising a strand reverse complementary to the wanted/desired RNA molecule. The “extension reaction” can also be designated as “reverse transcription” herein. In some embodiments, the wanted/desired RNA molecules are miRNA or isomiR molecules and the extension reaction is a reverse transcription reaction comprising a reverse transcriptase, whereby DNA (in particular cDNA) copies of the wanted/desired RNA molecules are made. In certain embodiments, the extension reaction is a reverse transcription reaction comprising a polymerase, such as a reverse transcriptase.

The term “reverse transcriptase”, as used herein, refers to any enzyme having reverse transcriptase activity. In particular, the term “reverse transcriptase”, as used herein, refers to an enzyme used to generate DNA (cDNA) from wanted/desired RNA molecules in a process termed reverse transcription. The reverse transcriptase has an RNA-dependent DNA polymerase activity. By means of this activity, a hybrid double strand of RNA and DNA is first built up after presentation of a single-stranded RNA by linking complementary paired DNA building blocks (deoxyribonucleotides). Afterwards, its RNA portion is largely degraded by means of an RNase H activity of a special section of the protein. The remaining DNA single strand is finally completed to the DNA double strand, catalyzed by an additional inherent DNA-dependent DNA polymerase activity of reverse transcriptase. To initiate reverse transcription, the reverse transcriptase requires a primer which serves as a starting point for the reverse transcriptase to synthesize a new strand. This primer is also called RT-primer sequence. The RT-primer can be reverse complementary to the wanted/desired RNA molecule. If the wanted/desired RNA molecule is flanked by a 5′adapter and a 3′adapter, the sequence of the RT-primer usually depends on the 3′ adapter sequence. In one preferred embodiment, the reverse transcriptase is selected from the group consisting of AMV (Avian myeloblastosis virus) reverse transcriptase, MoLV (Moloney murine leukemia virus) reverse transcriptase or Tth (Thermus thermopylus) DNA polymerase and their derivates.

In this respect, it should be noted that the first strand cDNA molecules may be used as templates in PCR experiments, e.g. qPCR experiments, to check the efficiency of the blocking oligonucleotides in inhibiting cDNA synthesis from unwanted RNA molecules to which the blocking oligonucleotides are reverse complementary. Briefly, an increase in Ct of amplifying a cDNA reverse transcribed from an unwanted RNA molecule when a blocking oligonucleotide is used during reverse transcription compared with when no blocking oligonucleotide is used during reverse transcription indicates that the blocking oligonucleotide is effective in inhibiting cDNA synthesis from the unwanted RNA molecule. The increase in Ct may be compared with that of another treatment (e.g., a commercially available treatment) to demonstrate equivalent to or improvement over the other treatment.

In certain embodiments, the Ct value of amplifying a cDNA reverse transcribed from an unwanted RNA molecule when a blocking oligonucleotide is used during reverse transcription is at least 2 times, at least 2.5 times, at least 3 times, or at least 4 times as much as the Ct value when no blocking oligonucleotide is used during reverse transcription.

The percentage of total reads that are derived from an unwanted RNA molecule when a blocking oligonucleotide is used during reverse transcription according to the present disclosure may be at most 5%, at most 4%, at most 3%, at most 2%, at most 1%, at most 0.8%, at most 0.6%, at most 0.5%, at most 0.4%, at most 0.3%, at most 0.2%, at most 0.1%, at most 0.05%, or 0%.

The ratio of the percentage of total reads that are derived from an unwanted RNA molecule when a blocking oligonucleotide is used during reverse transcription to that when no blocking oligonucleotide is used may be at most 0.2, at most 0.15, at most 0.1, at most 0.08, at most 0.06, at most 0.05, at most 0.04, at most 0.03, at most 0.02, or 0%.

Due to the process of reverse transcription, cDNA products of wanted/desired RNA molecules are produced. The method of producing a cDNA library from wanted/desired RNA molecules in an RNA sample described herein preferably further comprises the step of amplifying the cDNA products. Amplification is particularly carried out using a polymerase chain reaction (PCR). The PCR may be selected from the group consisting of real-time PCR (quantitative PCR or qPCR), preferably Taq-man qPCR, multiplex PCR, nested PCR, high fidelity PR, fast PCR, hot start PCR, and GC-rich PCR. The terms “amplicon” and “amplification product”, as used herein, generally refer to the product of an amplification reaction. An amplicon may be double-stranded or single-stranded, and may include the separated component strands obtained by denaturing a double-stranded amplification product. In certain embodiments, the amplicon of one amplification cycle can serve as a template in a subsequent amplification cycle.

The term “amplifying”, as used herein, refers to any means by which at least a part of wanted/desired RNA molecules is reproduced, typically in a template-dependent manner, including without limitation, a broad range of techniques for amplifying nucleic acid sequences, either linearly or exponentially. Any of several methods can be used to amplify the wanted/desired RNA molecules. Any in vitro means for multiplying the copies of a target sequence of nucleic acid can be utilized. These include linear, logarithmic, or other amplification methods. Exemplary methods include polymerase chain reaction (PCR), isothermal procedures (using one or more RNA polymerases, strand displacement, partial destruction of primer molecules, ligase chain reaction (LCR), Q RNA replicase systems, RNA transcription-based systems (e.g., TAS, 3SR), or rolling circle amplification (RCA).

In the context of the present invention, the cDNAs of the wanted/desired RNA molecules are amplified, in particular to produce a cDNA library of the wanted/desired RNA molecules in an RNA sample.

The term “cDNA library”, as used herein, refers to a collection of cDNA sequences that are complementary to the wanted/desired RNA molecules, in particular small non-coding wanted/desired RNA molecules, comprised in an RNA sample. A cDNA library, thus, reflects the composition of the wanted/desired RNA molecules, in particular small non-coding wanted/desired RNA molecules, comprised in an RNA sample. The RNA sample may comprise wanted/desired RNA molecules, in particular small non-coding wanted/desired RNA molecules, isolated from organisms, tissues, cells, or bodily fluids such as blood. The preparation of a cDNA library allows to examine the distribution of the wanted/desired RNA molecules, in particular small non-coding wanted/desired RNA molecules, comprised in an RNA sample. The preparation of a cDNA library further allows to obtain expression profiles of the wanted/desired RNA molecules, in particular small non-coding wanted/desired RNA molecules, comprised in an RNA sample.

Thus, further disclosed herein is a method of determining a profile of wanted/desired RNA molecules in an RNA sample. Said method comprises the sequencing of the double stranded cDNA products/cDNA library produced from the wanted/desired RNA molecules comprised in an RNA sample. Preferably the sequencing is next generation sequencing. By sequencing the double stranded cDNA products/cDNA library, the level of the wanted/desired RNA molecules in the RNA sample may be, e.g. quantitatively, be determined.

The term “level”, as used herein, refers to an amount (measured for example in grams, mole, or ion counts) or concentration (e.g. absolute or relative concentration, e.g. reads per million (RPM) or NGS counts) of an RNA molecule. The term “level”, as used herein, also comprises scaled, normalized, or scaled and normalized amounts or values. In particular, the level of the RNA molecule is determined by sequencing, preferably next generation sequencing (e.g. ABI SOLID, Illumina Genome Analyzer, Roche 454 GS FL, BGISEQ), nucleic acid hybridization (e.g. microarray or beads), nucleic acid amplification (e.g. PCR, RT-PCR, qRT-PCR, or high-throughput RT-PCR), polymerase extension, mass spectrometry, flow cytometry (e.g. LUMINEX), or any combination thereof. Specifically, the level of the RNA molecule is the expression level of said RNA molecule.

The term “next generation sequencing (NGS)” as used herein, refers to a new method for sequencing nucleotide sequences at high speed and at low cost. Next-generation sequencing (NGS) is, thus, a high-throughput methodology that enables rapid sequencing of the base pairs in DNA or RNA samples. Supporting a broad range of applications, including gene expression profiling, chromosome counting, detection of epigenetic changes, and molecular analysis, NGS is driving discovery and enabling the future of personalized medicine. NGS is also known as second generation sequencing (SGS) or massively parallel sequencing (MPS).

Residues in two or more polynucleotides are said to “correspond” to each other if the residues occupy an analogous position in the polynucleotide structures. It is well known in the art that analogous positions in two or more polynucleotides can be determined by aligning the polynucleotide sequences based on nucleic acid sequence or structural similarities. Such alignment tools are well known to the person skilled in the art and can be, for example, obtained on the World Wide Web, for example, ClustalW or Align using standard settings, preferably for Align EMBOSS::needle, Matrix: Blosum62, Gap Open 10.0, Gap Extend 0.5.

In the context of the present invention, the term “kit of parts (in short: kit)” is understood to be any combination of at least some of the components identified herein, which are combined, coexisting spatially, to a functional unit, and which can contain further components.

### EMBODIMENTS OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

The present inventors have developed new blocking oligonucleotides and methods wherein said blocking oligonucleotides are used. In this way, any abundant small RNA of choice can be targeted and its reverse transcription can be inhibited/prevented which in turn significantly decreases its presence in the NGS datasets. Accordingly, less abundant small RNA species can accurately be detected and quantified in a fast, easy, and cost-effective way.

Thus, in a first aspect, the present invention relates to a blocking oligonucleotide comprising one or more modified ribonucleotides and/or one or more locked nucleotides.

Particularly, the present invention relates to (i) a blocking oligonucleotide comprising one or more modified ribonucleotides, (ii) a blocking oligonucleotide comprising one or more locked nucleotides, or (iii) a blocking oligonucleotide comprising one or more modified ribonucleotides and one or more locked nucleotides.

In one embodiment, the blocking oligonucleotide is reverse complementary to at least a (core) region of an unwanted RNA molecule. In one particular embodiment, the blocking oligonucleotide is reverse complementary to at least a region at the 3′end of the unwanted RNA molecule. In one specific embodiment, the blocking oligonucleotide is reverse complementary to the entire length of the (core region of the) unwanted RNA molecule.

In one embodiment, the one or more modified ribonucleotides and/or the one or more locked nucleotides increase the binding of the blocking oligonucleotide to at least a (core) region of an unwanted RNA molecule. In one specific embodiment, the one or more modified ribonucleotides and/or the one or more locked nucleotides increase the binding of the blocking oligonucleotide to the entire length of the (core region of the) unwanted RNA molecule.

Due to this binding, the blocking oligonucleotide is able to inhibit cDNA synthesis using the (core) region of the unwanted RNA molecule. The formed duplex between the unwanted RNA molecule and the blocking oligonucleotide makes a reverse transcription reaction inefficient as the reverse transcriptase requires a single stranded RNA molecule as template. In addition, the binding of the blocking oligonucleotide to the region of the unwanted RNA molecule increases the melting temperature (Tm) of the duplex formed. This duplex is not destroyed during the reverse transcription reaction as the melting temperature (Tm) of the duplex formed is higher than the temperature at which the reverse transcription reaction is carried out, which is typically higher than 60° C.

Especially, the one or more modified ribonucleotides and/or the one or more locked nucleotides comprised in the blocking oligonucleotide increase the binding between the blocking oligonucleotide and a (core) region of an unwanted RNA molecule compared to an oligonucleotide with the same sequence but without the one or more modified ribonucleotides and/or one or more locked nucleotides if they increase the melting temperature (Tm) of the duplex formed between the blocking oligonucleotide comprising the one or more modified ribonucleotides and/or the one or more locked nucleotides and the (core) region of the unwanted RNA molecule compared to the melting temperature (Tm) of the duplex formed between the oligonucleotide with the same sequence but without the one or more modified ribonucleotides and/or the one or more locked nucleotides and the (core) region of the unwanted RNA molecule measured under the same conditions.

The Tm of duplexes formed between the blocking oligonucleotides as described herein and regions of unwanted RNA molecules to which the blocking oligonucleotides are reverse complementary range from between 4° and 85° C., preferably from between 6° and 75° C., as predicted by the freely available online tools designed to estimate such Tm.

Specifically, the unwanted RNA molecule is an abundant RNA molecule. Preferably, the unwanted RNA molecule is an abundant non-coding RNA molecule having a length of <200 ribonucleotides. More preferably, the unwanted RNA molecule is an abundant miRNA, miRNA isoform (isomiR), Y-RNA, or Y-RNA fragment, that is a not full-length Y-RNA. Alternatively, the unwanted RNA molecule is an abundant small nucleolar ribonucleic acid (snoRNA), ribosomal ribonucleic acid (rRNA) fragment, that is not full-length rRNA, or transfer RNA (tRNA). More specifically, the unwanted RNA molecule is an abundant miRNA, miRNA isoform (isomiR), Y-RNA, Y-RNA fragment, that is not full-length Y-RNA, snoRNA, rRNA fragment, that is not full-length rRNA, or tRNA.

Even more preferably, the unwanted RNA molecule is an abundant miRNA or miRNA isoform (isomiR). An abundant molecule is an RNA entity that consumes more than 0.05% of total processed reads in a small RNA NGS experiment. The skilled person is able to determine the abundance of an RNA molecule, e.g. in an NGS experiment.

In one embodiment, the one or more modified ribonucleotides are comprised in the 5′terminal nucleotide sequence and/or in the 3′terminal nucleotide sequence of the blocking oligonucleotide.

In one embodiment, the one or more locked nucleotides are comprised in the central nucleotide sequence of the blocking oligonucleotide.

Thus, in one particular embodiment, the blocking oligonucleotide comprises in the following order from 5′ to 3′:
(i) a 5′terminal nucleotide sequence comprising one or more (consecutive) modified ribonucleotides,
(ii) a central nucleotide sequence comprising one or more (non-consecutive) locked nucleotides, and
(ii) a 3′terminal nucleotide sequence comprising one or more (consecutive) modified ribonucleotides.

In one specific embodiment, the one or more locked nucleotides are not comprised in a stretch of 3 or more, e.g. 4, 5, or 6, G/C.

In one specific embodiment,
the locked nucleotides are not arranged in a consecutive order, or
each locked nucleotide is followed by at least one nucleotide which is not a locked nucleotide.

In one specific embodiment, the blocking oligonucleotide comprises between 3 and 10, preferably between 3 and 6, and more preferably between 4 and 5, e.g. 3, 4, 5, 6, 7, 8, 9, or 10, locked nucleotides.

LNA nucleotides can be mixed with DNA or RNA residues in the oligonucleotide and hybridize with DNA or RNA according to Watson-Crick base-pairing rules. The locked ribose conformation enhances base stacking and backbone pre-organization. This significantly increases the hybridization properties (melting temperature) of the blocking oligonucleotide. An increase in the duplex melting temperature of between 2 and 8° C., preferably of between 4 and 6° C., can be reached per LNA nucleotide when incorporated into the blocking oligonucleotide.

Particularly, the locked nucleotide is an LNA-ribonucleotide or an LNA-deoxyribonucleotide. More particularly, the locked nucleotide is selected from the group consisting of LNA-adenine, LNA-guanine, LNA-cytosine, and LNA-thymine.

In one another specific embodiment, the modified ribonucleotides which are comprised in the 5′terminal nucleotide sequence and/or the modified ribonucleotides which are comprised in the 3′terminal nucleotide sequence of the blocking oligonucleotide are arranged in a consecutive order.

In one another specific embodiment, the blocking oligonucleotide comprises between 4 and 22, preferably between 4 and 16, and more preferably between 6 and 10, e.g. 4, 5, 6, 7, 8, 9, 10, 11, 12, 13, 14, 15, 16, 17, 18, 19, 20, 21, or 22, modified ribonucleotides.

For example, the blocking oligonucleotide comprises between 3 to 5, e.g. 3, 4, or 5, (consecutive) modified ribonucleotides within the 5′terminal nucleotide sequence and/or between 3 to 5, e.g. 3, 4, or 5, (consecutive) modified ribonucleotides within the 3′terminal nucleotide sequence.

Particularly, the modified ribonucleotide(s) is (are) 2′-o-methyl ribonucleotide(s).

An increase in the duplex melting temperature of between 1 and 8° C., preferably of between 3 and 5° C., can be reached per 2′-o-methyl ribonucleotide when incorporated into the blocking oligonucleotide.

In one preferred embodiment, the blocking oligonucleotide comprises between 3 and 10, e.g. 3, 4, 5, 6, 7, 8, 9, or 10, locked nucleotides, wherein the locked nucleotides are not arranged in a consecutive order or wherein each locked nucleotide is followed by at least one nucleotide which is not a locked nucleotide.

Particularly, the locked nucleotides are LNA-ribonucleotides or LNA-deoxyribonucleotides. More particularly, the locked nucleotides are selected from the group consisting of LNA-adenine, LNA-guanine, LNA-cytosine, and LNA-thymine.

The blocking oligonucleotide of Version 1 designed by the present inventors is an example of this specific blocking oligonucleotide type (see experimental section).

In one alternative preferred embodiment, the blocking oligonucleotide consists of between 16 and 22, e.g. 16, 17, 18, 19, 20, 21, or 22, modified ribonucleotides, wherein the modified ribonucleotides are arranged in a consecutive order.

Particularly, the modified ribonucleotides are 2′-o-methyl ribonucleotides.

The blocking oligonucleotide of Version 2 designed by the present inventors is an example of this specific blocking oligonucleotide type (see experimental section).

In one more preferred embodiment, the blocking oligonucleotide comprises
(i) a 5′terminal nucleotide sequence comprising between 3 to 5, e.g. 3, 4, or 5, (consecutive) modified ribonucleotides,
(ii) a central nucleotide sequence comprising between 4 and 5, e.g. 4 or 5, (non-consecutive) locked nucleotides, and
(iii) a 3′terminal nucleotide sequence comprising between 3 to 5, e.g. 3, 4, or 5, (consecutive) modified ribonucleotides.

Particularly, the locked nucleotides are LNA-ribonucleotides or LNA-deoxyribonucleotides and/or the modified ribonucleotides are 2′-o-methyl ribonucleotides. More particularly, the locked nucleotides are selected from the group consisting of LNA-adenine, LNA-guanine, LNA-cytosine, and LNA-thymine and/or the modified ribonucleotides are 2′-o-methyl ribonucleotides.

The blocking oligonucleotide of Version 3 designed by the present inventors is an example of this specific blocking oligonucleotide type (see experimental section).

It should be noted that all blocking oligonucleotides as described herein do not comprise a modification at their 3′end. The 3′ modification renders the blocking oligonucleotide incapable of being extended by a polymerase or reverse transcriptase.

In one embodiment, the blocking oligonucleotide has a length of between 16 and 22, e.g. 16, 17, 18, 19, 20, 21, or 22, nucleotides.

The blocking oligonucleotide designed by the present inventors allows the detection of an unwanted miRNA. The blocking oligonucleotide designed by the present inventors also allows the detection of all isomiRs of said unwanted miRNA. This is possible as the blocking oligonucleotide designed by the present inventors is reverse complementary to the core sequence of the miRNA as well as to all isomiRs of said miRNA.

The region of the unwanted RNA molecule to which the blocking oligonucleotide is reverse complementary may be at least 10 nucleotides in length, such as at least 11, 12, 13, 14, 15, 16, 17, 18, 19, 20, 21 nucleotides in length or 22 nucleotides in length. Such a region may be at most 30 nucleotides in length, such as at most 30, 29, 28, 27, 26, 25, 24, 23, or 22 nucleotides in length. In one preferred embodiment, the blocking oligonucleotide is reverse complementary to a region of between 14 and 20 nucleotides in length, e.g. 14, 15, 16, 17, 18, 19, 20, 21, or 22 nucleotides in length, of an unwanted RNA molecule. Preferably, the blocking oligonucleotide is reverse complementary to a (core) region of between 14 and 20 nucleotides in length, e.g. 14, 15, 16, 17, 18, 19, or 20 nucleotides in length, of an unwanted RNA molecule. More preferably, the blocking oligonucleotide is reverse complementary to a (core) region of between 16 and 22 nucleotides in length, e.g. 16, 17, 18, 19, 20, 21, or 22 nucleotides in length, of an unwanted RNA molecule.

In one still even more preferred embodiment, the blocking oligonucleotide is reverse complementary to at least a (core) region of an unwanted RNA molecule having a nucleotide sequence selected from the group consisting of SEQ ID NO: 34 to SEQ ID NO: 66 or to at least a (core) region of unwanted RNA molecule isomers thereof. Here the unwanted RNA molecule is a miRNA or a Y-RNA molecule. Alternatively, the blocking oligonucleotide is reverse complementary to at least a (core) region of an unwanted RNA molecule having a nucleotide sequence selected from the group consisting of SEQ ID NO: 123 to SEQ ID NO: 169 or to at least a (core) region of unwanted RNA molecule isomers thereof. Specifically, the blocking oligonucleotide is reverse complementary to at least a (core) region of an unwanted RNA molecule having a nucleotide sequence selected from the group consisting of SEQ ID NO: 34 to SEQ ID NO: 66 or to at least a (core) region of unwanted RNA molecule isomers thereof and SEQ ID NO: 123 to SEQ ID NO: 169 or to at least a (core) region of unwanted RNA molecule isomers thereof. More specifically, the blocking oligonucleotide is reverse complementary to at least a (core) region of an unwanted RNA molecule having a nucleotide sequence selected from the group consisting of SEQ ID NO: 34 to SEQ ID NO: 66 and SEQ ID NO: 123 to SEQ ID NO: 169.

Particularly, the blocking oligonucleotide is reverse complementary to at least a (core) region of an unwanted miRNA molecule having a nucleotide sequence selected from the group consisting of SEQ ID NO: 34 to SEQ ID NO: 36, SEQ ID NO: 38 to SEQ ID NO: 53, SEQ ID NO: 55 to SEQ ID NO: 57, and SEQ ID NO: 59 to SEQ ID NO: 66 or to at least a (core) region of unwanted isomiR molecules thereof.

Particularly, the blocking oligonucleotide is reverse complementary to at least a (core) region of an unwanted Y-RNA molecule having a nucleotide sequence selected from the group consisting of SEQ ID NO: 37, SEQ ID NO: 54 and SEQ ID NO: 58.

In one most preferred embodiment, the blocking oligonucleotide has a nucleotide sequence selected from the group consisting of SEQ ID NO: 1 to SEQ ID NO: 33 or is a variant of this nucleotide sequence. Here the blocking nucleotide is a miRNA/isomiR blocking oligonucleotide or a Y-RNA molecule blocking oligonucleotide. Alternatively, the blocking oligonucleotide has a nucleotide sequence selected from the group consisting of SEQ ID NO: 76 to SEQ ID NO: 122 or is a variant of this nucleotide sequence. Specifically, the blocking oligonucleotide has a nucleotide sequence selected from the group consisting of SEQ ID NO: 1 to SEQ ID NO: 33 or is a variant of this nucleotide sequence and SEQ ID NO: 76 to SEQ ID NO: 122 or is a variant of this nucleotide sequence. More specifically, the blocking oligonucleotide has a nucleotide sequence selected from the group consisting of SEQ ID NO: 1 to SEQ ID NO: 33 and SEQ ID NO: 76 to SEQ ID NO: 122.

Particularly, the miRNA/isomiR blocking oligonucleotide has a nucleotide sequence selected from the group consisting of SEQ ID NO: 1 to SEQ ID NO: 3, SEQ ID NO: 5 to SEQ ID NO: 20, SEQ ID NO: 22 to SEQ ID NO: 24 and, SEQ ID NO: 26 to SEQ ID NO: 33 or is a variant of this nucleotide sequence.

Particularly, the Y-RNA blocking oligonucleotide has a nucleotide sequence selected from the group consisting of SEQ ID NO: 4, SEQ ID NO: 21 and SEQ ID NO: 25 or is a variant of this nucleotide sequence.

The blocking oligonucleotide variant as described above has a nucleotide sequence having at least 80%, preferably 85%, more preferably 90%, even more preferably 95%, and still even more preferably 99%, e.g. 80, 81, 82, 83, 84, 85, 86, 87, 88, 89, 90, 91, 92, 93, 94, 95, 96, 97, 98, or 99%, sequence identity to the nucleotide sequence according to SEQ ID NO: 1 to SEQ ID NO: 33 or SEQ ID NO: 76 to SEQ ID NO: 122. Such a blocking oligonucleotide variant still comprises the modified ribonucleotides. In addition, such a blocking oligonucleotide variant is still LNA enhanced. Moreover, such a blocking oligonucleotide variant is still capable of inhibiting cDNA synthesis. The skilled person can readily assess whether a blocking oligonucleotide variant is still capable of inhibiting cDNA synthesis. For example, the experimental section provides sufficient information in this respect.

The blocking oligonucleotide designed by the present inventors allows the detection of an unwanted miRNA. The blocking oligonucleotide designed by the present inventors also allows the detection of all isomiRs of said miRNA. This is possible as the blocking oligonucleotide designed by the present inventors is reverse complementary to the core sequence of the miRNA as well as to all isomiRs of said miRNA.

In a second aspect, the present invention relates to a set comprising at least two blocking oligonucleotides, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 blocking oligonucleotides, or e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47 blocking oligonucleotides, or e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 blocking oligonucleotides, according to the first aspect.

Particularly, the at least two blocking oligonucleotides, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 blocking oligonucleotides, or e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47 blocking oligonucleotides, or e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 blocking oligonucleotides, differ from each other in their nucleotide sequence. More particularly, the at least two blocking oligonucleotides, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 blocking oligonucleotides, or e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or 47 blocking oligonucleotides, or e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 blocking oligonucleotides, are reverse complementary to at least a (core) region of different unwanted RNA molecules.

In one most preferred embodiment, the set comprises blocking oligonucleotides having a nucleotide sequence according to SEQ ID NO: 1 to SEQ ID NO: 33. Alternatively, the set comprises blocking oligonucleotides having a nucleotide sequence according to SEQ ID NO: 76 to SEQ ID NO: 122. Specifically, the set comprises blocking oligonucleotides having a nucleotide sequence according to SEQ ID NO: 1 to SEQ ID NO: 33 and SEQ ID NO: 76 to SEQ ID NO: 122. This set allows the inhibition of specific unwanted miRNA and/or isomiR molecules as well as Y-RNA molecules. A set specific for the inhibition of defined unwanted miRNA and/or isomiR molecules, particularly comprises the blocking oligonucleotides having a nucleotide sequence according to SEQ ID NO: 1 to SEQ ID NO: 3, SEQ ID NO: 5 to SEQ ID NO: 20, SEQ ID NO: 22 to SEQ ID NO: 24 and, SEQ ID NO: 26 to SEQ ID NO: 33. In addition, a set specific for the inhibition of defined unwanted Y-RNA molecules, particularly comprises the blocking oligonucleotides having a nucleotide sequence according to SEQ ID NO: 4, SEQ ID NO: 21 and SEQ ID NO: 25. Moreover, a set specific for the inhibition of defined unwanted snoRNA molecules, particularly comprises the blocking oligonucleotides against snoRNAs as shown in the table in the experimental section, a set specific for the inhibition of defined unwanted rRNA fragments, particularly comprises the blocking oligonucleotides against rRNA fragments as shown in the table in the experimental section, and/or a set specific for the inhibition of defined unwanted tRNA molecules, particularly comprises the blocking oligonucleotides against tRNAs as shown in the table in the experimental section.

In a third aspect, the present invention relates to a method for inhibiting cDNA synthesis of one or more unwanted RNA molecules in an RNA sample during reverse transcription comprising the steps of:
(i) providing a mixture containing an RNA sample comprising one or more desired RNA molecules and one or more unwanted RNA molecules and one or more blocking oligonucleotides according to the first aspect (or blocking oligonucleotides comprised in the set according to the second aspect), wherein the one or more blocking oligonucleotides are annealed to the one or more unwanted RNA molecules, and
(ii) reverse transcribing the one or more desired RNA molecules, thereby obtaining one or more cDNA products from said desired RNA molecule(s) and inhibiting cDNA synthesis of the one or more unwanted RNA molecules.

In a fourth aspect, the present invention relates to a method for (improving) cDNA synthesis of one or more desired RNA molecules in an RNA sample during reverse transcription comprising the steps of:
(i) providing a mixture containing an RNA sample comprising one or more desired RNA molecules and one or more unwanted RNA molecules and one or more blocking oligonucleotides according to the first aspect (or blocking oligonucleotides comprised in the set according to the second aspect), wherein the one or more blocking oligonucleotides are annealed to the one or more unwanted RNA molecules, and
(ii) reverse transcribing the one or more desired RNA molecules, thereby obtaining one or more cDNA products from said desired RNA molecule(s) and inhibiting cDNA synthesis of the one or more unwanted RNA molecules.

The following description applies to the third aspect as well as fourth aspect of the present invention:

In one embodiment (of the third and fourth aspect), the annealing of the one or more blocking oligonucleotides to the one or more unwanted RNA molecules has been achieved by denaturating and subsequently cooling down the one or more blocking oligonucleotides and the one or more unwanted RNA molecules contained in the mixture.

It is preferred that the denaturation has been carried out at between 75 and 90° C., e.g. 75, 76, 77, 78, 79, 80, 81, 82, 83, 84, 85, 86, 87, 88, 89, or 90° C., particularly for between 1 and 5 minutes, e.g. 1, 2, 3, 4, or 5 minutes.

It is also preferred that the cooling down has been carried out to between 2° and 30° C., e.g. 20, 21, 22, 23, 24, 25, 26, 27, 28, 29, or 30° C., particularly at a rate of between 0.1 and 2° C./s, e.g. 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2° C./s.

It is more preferred that the denaturation has been carried out at between 75 and 90° C., e.g. 75, 76, 77, 78, 79, 80, 81, 82, 83, 84, 85, 86, 87, 88, 89, or 90° C., particularly for between 1 and 5 minutes, e.g. 1, 2, 3, 4, or 5 minutes, and that the cooling down has been carried out to between 20 and 30° C., e.g. 20, 21, 22, 23, 24, 25, 26, 27, 28, 29, or 30° C., particularly at a rate of between 0.1 and 2° C./s, e.g. 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, or 2° C./s.

Specifically, the one more desired RNA molecules, the one or more unwanted RNA molecules and the one or more blocking oligonucleotides comprised in the mixture have been denatured and subsequently cooled down, wherein the denaturation and the subsequent cooling down results in the annealing of the one or more blocking oligonucleotides to the one or more unwanted RNA molecules. As to the specific denaturation and cooling down conditions, it is referred to the explanations above.

In one another embodiment (of the third and fourth aspect), the mixture (provided in step (i)) has been produced by mixing
the RNA sample comprising one or more desired RNA molecules and one or more unwanted RNA molecules with the one or more blocking oligonucleotides, or
the one or more blocking oligonucleotides with the RNA sample comprising one or more desired RNA molecules and one or more unwanted RNA molecules.

In one preferred embodiment (of the third and fourth aspect), the reverse transcription of the one or more desired RNA molecules is carried out by
(iia) annealing a primer for reverse transcription (RT) primer with the one or more desired RNA molecules, and
(iib) reverse transcribing the one or more desired RNA molecules by using/with a reverse transcriptase (RT).

It is more preferred that said annealing is carried out at between 25 and 60° C., e.g. 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., particularly for between 1 and 10 minutes, e.g. 1, 2, 3, 4, 5, 6, 7, 8, 9, or 10 minute(s). It is even more preferred that said annealing is carried out at between 37 and 56° C., e.g. 37, 38, 39, 40, 41, 42, 43, 44, 45, 46, 47, 48, 49, 50, 51, 52, 53, 54, 55, or 56° C., particularly for between 3 and 6 minutes, e.g. 3, 4, 5, or 6 minutes.

It is also more preferred that said reverse transcribing is carried out at between 37 and 72° C., e.g. 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72° C., particularly for between 1 and 90 minutes, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 minute(s). It is also even more preferred that said reverse transcribing is carried out at between 4° and 60° C., e.g. 40, 41, 42, 43, 44, 45, 46, 47, 48, 49, 50, 51, 52, 53, 54, 55, 56, 57, 58, 59, or 60° C., particularly for between 10 and 35 minutes, e.g. 10, 11, 12, 13, 14, 15, 16, 17, 18, 19, 20, 21, 22, 23, 24, 25, 26, 27, 28, 29, 30, 31, 32, 33, 34, or 35 minutes.

It is still even more preferred that said annealing is carried out at between 25 and 60° C., e.g. 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60° C., particularly for between 1 and 10 minutes, e.g. 1, 2, 3, 4, 5, 6, 7, 8, 9, or 10 minute(s) and that said reverse transcribing is carried out at between 37 and 72° C., e.g. 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72° C., particularly for between 1 and 90 minutes, e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 minute(s).

The reverse transcriptase may be selected from the group consisting of AMV (Avian myeloblastosis virus) reverse transcriptase, MoLV (Moloney murine leukemia virus) reverse transcriptase or Tth (Thermus thermopylus) DNA polymerase and their derivates.

To initiate reverse transcription, the reverse transcriptase requires a primer which serves as a starting point for the reverse transcriptase to synthesize a new strand. This primer is also called RT-primer. The RT-primer can be reverse complementary to the desired RNA molecule. If the desired RNA molecule is flanked by a 5′adapter and a 3′adapter, the sequence of the RT-primer can depend on the 3′ adapter sequence.

Particularly, the RNA sample is a biological sample or prepared/obtained from a biological sample. More particularly, the biological sample is a blood sample. Even more particularly, the blood sample is a whole blood or a blood fraction, specifically blood cells (e.g. erythrocytes, leukocytes, and/or thrombocytes), serum, or plasma. The RNA sample may also contain total RNA.

It is further clear that the RNA sample specifically encompasses miRNAs and/or miRNA isoforms (isomiR) as wanted/unwanted RNA molecules. Said miRNAs and/or miRNA isoforms (isomiR) may be present as single stranded RNA molecules. Said miRNAs and/or miRNA isoforms (isomiR) may also be present attached to/linked to/flanked by adapter molecules. Especially, the adapter molecules are ligated to the 5′end of the miRNAs or miRNA isoforms (isomiR) (5′adapter) and to the 3′end of the miRNAs or miRNA isoforms (isomiR) (3′adapter). In other words, the miRNAs and/or miRNA isoforms (isomiR) as wanted/unwanted RNA molecules are flanked by 5′adapter and 3′adapter molecules. The adapters, hybridized to the 5′ and the 3′end of the miRNAs and/or miRNA isoforms (isomiR), enable the formation of ligated RNA molecules that can be used as templates for cDNA synthesis. In this way, not only miRNAs but also their variants, i.e. isomiRs, can be detected, which improves different molecular techniques such as next generation sequencing (NGS), cDNA library constructions, and diverse diagnostic methods.

The inhibition of cDNA synthesis of an unwanted RNA molecule means that the amount of single stranded or double stranded cDNA generated using the unwanted RNA molecule as a template during reverse transcription is reduced at a statistically significant degree under a modified condition (e.g. in the presence of a blocking oligonucleotide comprising one or more modified ribonucleotides and/or one or more locked nucleotides) compared to the amount of single stranded or double stranded cDNA generated during reverse transcription under a reference condition (e.g. in the absence of a blocking oligonucleotide comprising one or more modified ribonucleotides and/or one or more locked nucleotides). The reduction in the amount of synthesized cDNA may be measured using qPCR or transcriptome sequencing as disclosed herein. Particularly, the cDNA synthesis of an unwanted RNA molecule is in inhibited to at least 20%, 30%, 40%, 50%, 60%, 70%, 80%, 90%, 95%, 99%, or even 100%. The inhibition of cDNA synthesis of an unwanted RNA molecule may be referred to as depletion of the unwanted RNA molecule. Even though the unwanted RNA molecule is not physically removed from an initial RNA sample, the involvement of the unwanted RNA molecule in the downstream manipulation or analysis of the initial RNA sample is reduced or eliminated due to the inhibition of cDNA synthesis of the unwanted RNA molecule.

In turn, the cDNA synthesis of a desired RNA molecule is improved. The improvement in the amount of synthesized cDNA may be measured using qPCR or transcriptome sequencing as disclosed herein. Particularly, the cDNA synthesis of a desired RNA molecule is in improved by at least 20%, 30%, 40%, 50%, 60%, 70%, 80%, 90%, 95%, 99%, or even 100%.

Specifically, the unwanted RNA molecules are abundant RNA molecules. Preferably, the unwanted RNA molecules are abundant non-coding RNA molecules having a length of <200 ribonucleotides. More preferably, the unwanted RNA molecules are abundant miRNAs, miRNA isoforms (isomiRs), Y-RNAs, and/or Y-RNA fragments, that are a not full-length Y-RNAs. Alternatively, the unwanted RNA molecules are abundant small nucleolar ribonucleic acids (snoRNAs), ribosomal ribonucleic acid (rRNA) fragments, that are not full-length rRNAs, and/or transfer RNAs (tRNAs). More specifically, the unwanted RNA molecules are abundant miRNAs, miRNA isoforms (isomiRs), Y-RNAs, Y-RNA fragments, that are not full-length Y-RNAs, snoRNAs, rRNA fragments, that are not full-length rRNAs, and/or tRNAs. Even more preferably, the unwanted RNA molecules are abundant miRNAs and/or miRNA isoforms (isomiRs). Abundant RNA molecules are RNA entities that consume more than 0.05% of total processed reads in a small RNA NGS experiment. The skilled person is able to determine the abundance of RNA molecules, e.g. in an NGS experiment.

In reverse, the desired RNA molecules are less abundant RNA molecules. Preferably, the desired RNA molecules are less abundant non-coding RNA molecules having a length of <200 ribonucleotides. More preferably, the desired RNA molecules are less abundant miRNAs, miRNA isoforms (isomiRs), Y-RNAs, and/or Y-RNA fragments, that are a not full-length Y-RNAs. Alternatively, the desired RNA molecules are less abundant small nucleolar ribonucleic acids (snoRNAs), ribosomal ribonucleic acid (rRNA) fragments, that are not full-length rRNAs, and/or transfer RNAs (tRNAs). More specifically, the desired RNA molecules are less abundant miRNAs, miRNA isoforms (isomiRs), Y-RNAs, Y-RNA fragments, that are not full-length Y-RNAs, snoRNAs, rRNA fragments, that are not full-length rRNAs, and/or tRNAs. Even more preferably, the desired RNA molecules are less abundant miRNAs and/or miRNA isoforms (isomiRs). Less abundant RNA molecules are RNA entities that consume equal to or less than 0.05% of total processed reads in a small RNA NGS experiment. The skilled person is able to determine the abundance of RNA molecules, e.g. in an NGS experiment.

In a fifth aspect, the present invention relates to a method for producing one or more double stranded cDNA products from one or more desired RNA molecules comprising the steps of:
(i) carrying out the method according to the third or fourth aspect, and
(ii) amplifying the one or more cDNA products, thereby producing one or more double stranded cDNA products from one or more desired RNA molecules.

When with the above method at least two double stranded cDNA products from at least two desired RNA molecules are produced, a cDNA library is constructed.

In one preferred embodiment, the amplification is carried out using a polymerase chain reaction (PCR). In one more preferred embodiment, the PCR is selected from the group consisting of real-time PCR (quantitative PCR or qPCR), preferably Taq-man qPCR, multiplex PCR, nested PCR, high fidelity PR, fast PCR, hot start PCR, and GC-rich PCR.

It should be clear for a skilled person that the amplification is carried out with a Forward primer and a Reverse primer. The sequences of the Forward primer and a Reverse primer depend on the cDNA product used as a template. The skilled person can design a suitable Forward primer and a suitable Reverse primer which can be used in the above method without problems.

It is preferred that the Forward and Reverse primer comprises a Unique Dual Index (UDI). In this respect, it should be noted that UDI is an acronym for Unique Dual Index. With unique dual indexes (UDIs), each sample index is specific to a given sample library. All nucleic acids in a sample are labeled with the same sequence tag, and the resulting library is pooled with other libraries and sequenced in parallel in a single run.

Particularly, the amplification is carried out with a DNA polymerase. The DNA polymerase may be is selected from the group consisting of Taq DNA polymerase, Tth DNA polymerase and Pfu DNA polymerase and derivates thereof.

In a sixth aspect, the present invention relates to a method for determining a profile of one or more desired RNA molecules comprising the step of:
sequencing the one or more double stranded cDNA products from the one or more desired RNA molecules produced by the method according to the firth aspect.

When a cDNA library was the result of the method according to the fifth aspect of the present invention, this cDNA library is sequences with in the method according to the sixth aspect of the present invention.

Any sequencing technique known by the skilled person may be used. By sequencing the double stranded cDNA products (the cDNA library), the level of the desired RNAs in the RNA sample may be, e.g. quantitatively, determined. The level of the desired RNAs is specifically the expression level of said desired RNAs.

Preferably, the sequencing is next generation sequencing (NGS). Next generation sequencing is a new method for sequencing nucleotide sequences at high speed. Next-generation sequencing (NGS) is, thus, a high-throughput methodology that enables rapid sequencing of the base pairs in DNA or RNA samples. It supports molecular as well as diagnostic analysis by means of generating RNA expression data for wide range of RNAs that can be used for diagnostic and prognostic purposes.

Particularly, the profile is the expression profile of the one or more desired RNA molecules.

In a seventh aspect, the present invention relates to the use of the blocking oligonucleotide according to the first aspect for inhibiting cDNA synthesis of (an) unwanted RNA molecule(s) in an RNA sample during reverse transcription.

Specifically, the unwanted RNA molecules are abundant RNA molecules. Preferably, the unwanted RNA molecules are abundant non-coding RNA molecules having a length of <200 ribonucleotides. More preferably, the unwanted RNA molecules are abundant miRNAs, miRNA isoforms (isomiRs), Y-RNAs, and/or Y-RNA fragments, that are a not full-length Y-RNAs. Alternatively, the unwanted RNA molecules are abundant small nucleolar ribonucleic acids (snoRNAs), ribosomal ribonucleic acid (rRNA) fragments, that are not full-length rRNAs, and/or transfer RNAs (tRNAs). More specifically, the unwanted RNA molecules are abundant miRNAs, miRNA isoforms (isomiRs), Y-RNAs, Y-RNA fragments, that are not full-length Y-RNAs, snoRNAs, rRNA fragments, that are not full-length rRNAs, and/or tRNAs. Even more preferably, the unwanted RNA molecules are abundant miRNAs and/or miRNA isoforms (isomiRs). Abundant RNA molecules are RNA entities that consume more than 0.05% of total processed reads in a small RNA NGS experiment. The skilled person is able to determine the abundance of RNA molecules, e.g. in an NGS experiment.

As to preferred embodiments of the blocking oligonucleotide, unwanted RNA molecules, and RNA sample, it is referred to the first, third, and fourth aspect of the present invention.

In an eight aspect, the present invention relates to the use of the set comprising at least two oligonucleotides according to the second aspect for inhibiting cDNA synthesis of unwanted RNA molecules in an RNA sample during reverse transcription.

Specifically, the unwanted RNA molecules are abundant RNA molecules. Preferably, the unwanted RNA molecules are abundant non-coding RNA molecules having a length of <200 ribonucleotides. More preferably, the unwanted RNA molecules are abundant miRNAs, miRNA isoforms (isomiR), Y-RNAs, and/or Y-RNA fragments, that are a not full-length Y-RNAs. Alternatively, the unwanted RNA molecules are abundant small nucleolar ribonucleic acids (snoRNAs), ribosomal ribonucleic acid (rRNA) fragments, that are not full-length rRNAs, and/or transfer RNAs (tRNAs). More specifically, the unwanted RNA molecules are abundant miRNAs, miRNA isoforms (isomiRs), Y-RNAs, Y-RNA fragments, that are not full-length Y-RNAs, snoRNAs, rRNA fragments, that are not full-length rRNAs, and/or tRNAs. Even more preferably, the unwanted RNA molecules are abundant miRNAs and/or miRNA isoforms (isomiRs). Abundant RNA molecules are RNA entities that consume more than 0.05% of total processed reads in a small RNA NGS experiment. The skilled person is able to determine the abundance of RNA molecules, e.g. in an NGS experiment.

As to preferred embodiments of the blocking oligonucleotide, unwanted RNA molecules, and RNA sample, it is referred to the first, second, third, and fourth aspect of the present invention.

In a ninth aspect, the present invention relates to a kit comprising
the oligonucleotide according to the first aspect, or
the set comprising at least two oligonucleotides according to the second aspect.

In one preferred embodiment, the kit further comprises a reverse transcriptase. Particularly, the reverse transcriptase is selected from the group consisting of AMV (Avian myeloblastosis virus) reverse transcriptase, MoLV (Moloney murine leukemia virus) reverse transcriptase or Tth (Thermus thermopylus) DNA polymerase and their derivates.

To initiate reverse transcription, the reverse transcriptase requires a primer which serves as a starting point for the reverse transcriptase to synthesize a new strand. This primer is also called RT-primer. Thus, more particularly, the kit further comprises RT primer and/or buffer components to carry out reverse transcription.

The RT-primer can be reverse complementary to the desired RNA molecule. If the desired RNA molecule is flanked by a 5′adapter and a 3′adapter, the sequence of the RT-primer can depend on the 3′ adapter sequence.

In this case, the kit specifically further comprises instructions on how to carry out the method according to the third or fourth aspect of the present invention. The kit with this composition, specifically further allows to conduct the method according to the third or fourth aspect of the present invention.

In one (additional or alternative) preferred embodiment, the kit further comprises a DNA polymerase. Particularly, the DNA polymerase is selected from the group consisting of Taq DNA polymerase, Tth DNA polymerase and Pfu DNA polymerase and derivates thereof. More particularly, the kit further comprises Forward and Reverse primer and/or buffer components to carry out DNA amplification.

In this case, the kit specifically further comprises instructions on how to carry out the method according to the fifth aspect of the present invention. The kit with this composition, specifically further allows to conduct the method according to the fifth aspect of the present invention.

In one (additional or alternative) preferred embodiment, the kit further comprises means for sequencing (e.g. next generation sequencing), in particular of double stranded cDNA products (e.g. a cDNA library), such as instructions, necessary components, positive and negative controls. In this case, the kit preferably further comprises instructions on how to carry out the method according to sixth aspect. The kit with this composition preferably further allows to conduct the method according to the sixth aspect.

Especially, the kit is suitable/used for inhibiting cDNA synthesis of one or more unwanted RNA molecules in an RNA sample during reverse transcription. Alternatively, the kit is suitable/used for improving cDNA synthesis of one or more desired RNA molecules in an RNA sample during reverse transcription.

Particularly, the RNA sample is a biological sample or prepared/obtained from a biological sample. More particularly, the biological sample is a blood sample. Even more particularly, the blood sample is a whole blood or a blood fraction, specifically blood cells (e.g. erythrocytes, leukocytes, and/or thrombocytes), serum, or plasma. The RNA sample may also contain total RNA.

It is further clear that the RNA sample specifically encompasses miRNAs and/or miRNA isoforms (isomiR) as wanted/unwanted RNA molecules. Said miRNAs and/or miRNA isoforms (isomiR) may be present as single stranded RNA molecules. Said miRNAs and/or miRNA isoforms (isomiR) may also be present attached to/linked to/flanked by adapter molecules. Especially, the adapter molecules are ligated to the 5′end of the miRNAs or miRNA isoforms (isomiR) (5′adapter) and to the 3′end of the miRNAs or miRNA isoforms (isomiR) (3′adapter). In other words, the miRNAs and/or miRNA isoforms (isomiR) as wanted/unwanted RNA molecules are flanked by 5′adapter and 3′adapter molecules. The adapters, hybridized to the 5′ and the 3′end of the miRNAs and/or miRNA isoforms (isomiR), enable the formation of ligated RNA molecules that can be used as templates for cDNA synthesis. In this way, not only miRNAs but also their variants, i.e. isomiRs, can be detected, which improves different molecular techniques such as next generation sequencing (NGS), cDNA library constructions, and diverse diagnostic methods.

In view of the above, the kit comprises in one more preferred embodiment the following components:
the oligonucleotide according to the first aspect, or
the set comprising at least two blocking oligonucleotides according to the second aspect,
a reverse transcriptase, and
optionally RT-primer to carry out reverse transcription.

Particularly, the reverse transcriptase is selected from the group consisting of AMV (Avian myeloblastosis virus) reverse transcriptase, MoLV (Moloney murine leukemia virus) reverse transcriptase or Tth (Thermus thermopylus) DNA polymerase and their derivates.

Buffer components to carry out reverse transcription may further be part of the kit.

The kit comprises in one even more preferred embodiment the following components: the oligonucleotide according to the first aspect, or
the set comprising at least two blocking oligonucleotides according to the second aspect,
a reverse transcriptase,
a DNA polymerase, and
optionally RT-primer to carry out reverse transcription and/or Forward and Reverse primer to carry out DNA amplification.

Particularly, the reverse transcriptase is selected from the group consisting of AMV (Avian myeloblastosis virus) reverse transcriptase, MoLV (Moloney murine leukemia virus) reverse transcriptase or Tth (Thermus thermopylus) DNA polymerase and their derivates.

Particularly, the DNA polymerase is selected from the group consisting of Taq DNA polymerase, Tth DNA polymerase and Pfu DNA polymerase and derivates thereof.

Buffer components to carry out reverse transcription and/or DNA amplification may further be part of the kit.

The kit comprises in one still even more preferred embodiment the following components:
the oligonucleotide according to the first aspect, or
the set comprising at least two blocking oligonucleotides according to the second aspect, a reverse transcriptase,
a DNA polymerase,
means for sequencing (e.g. next generation sequencing), and
optionally RT-primer to carry out reverse transcription and/or Forward and Reverse primer to carry out DNA amplification.

Particularly, the reverse transcriptase is selected from the group consisting of AMV (Avian myeloblastosis virus) reverse transcriptase, MoLV (Moloney murine leukemia virus) reverse transcriptase or Tth (Thermus thermopylus) DNA polymerase and their derivates.

Particularly, the DNA polymerase is selected from the group consisting of Taq DNA polymerase, Tth DNA polymerase and Pfu DNA polymerase and derivates thereof.

Buffer components to carry out reverse transcription, DNA amplification, and sequencing (e.g. next generation sequencing) may further be part of the kit.

The kit may further comprise
(i) one or more containers for the different components of the kit, and/or
(ii) a data carrier.

Said data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. The access code may also allow access to an application software that causes a computer to perform tasks for computer users or a mobile app which is a software designed to run on smartphones and other mobile devices.

Said data carrier may further comprise information or instructions on how to carry out the methods according to the third to sixth aspect of the present invention.

Said kit may also comprise materials desirable from a commercial and user standpoint including a buffer(s), a reagent(s) and/or a diluent(s) which might be helpful to carry out the methods according to the third to sixth aspect of the present invention.

It should be noted that the blocking oligonucleotide having a nucleotide sequence selected from the group consisting of SEQ ID NO: 1 to SEQ ID NO: 33, as described herein, is reverse complementary to at least a (core) region of an unwanted RNA molecule having a nucleotide sequence selected from the group consisting of SEQ ID NO: 34 to SEQ ID NO: 66, respectively. In addition, the blocking oligonucleotide having a nucleotide sequence selected from the group consisting of SEQ ID NO: 76 to SEQ ID NO: 122, as described herein, is reverse complementary to at least a (core) region of an unwanted RNA molecule having a nucleotide sequence selected from the group consisting of SEQ ID NO: 123 to SEQ ID NO: 169, respectively.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following Figures are merely illustrative of the present invention and should not be construed to limit the scope of the invention as indicated by the appended claims in any way.

FIG. 1: qRT-PCR results of the first blocking test showing the relative expression of miR-16-5p. No blocking control is set to 1. The results indicated that the custom oligos can suppress the expression of miR-16-5p 10,000-fold or more.

FIG. 2: Normalized RPM values of mapped miRNAs are plotted. On the X-axis values of a no blocking control sample are plotted, while on the Y-axis values of the blocking oligo sample are plotted. The values are depicted as log 2 RPM. The target miR-16-5p miRNA is annotated.

FIG. 3: Normalized RPM values of all detected RNAs are plotted. On X-axis are values of a no blocking control sample, while on Y-axis is blocking oligo sample. The values are depicted as log 2 RPM. The RNAs with RPM lower than 1 are not depicted. The sequences (canonical miRNA and isomiRs) of miR-16-5p are depicted in black and annotated.

FIG. 4: Normalized RPM values of mapped miRNAs are plotted. On the Y-axis values of a no blocking control sample are plotted, while on the X-axis values of the sample from blocking oligo mixture experiment are plotted. The values are depicted as log 2 RPM. miRNAs that underwent blocking, either as targets or off-targets are annotated.

### EXAMPLES

The examples given below are for illustrative purposes only and do not limit the invention described above in any way.

### Introduction

Clinical peripheral blood samples drawn into PAXgene tubes can be used for the extraction of RNA. PAXgene RNA samples represent total RNA that was included in the cellular compartment (blood cells) and non-cellular compartment (plasma) of the blood. PAXgene RNA is typically isolated using silica bead-based procedure and stored at −80° C. after elution.

Due to the indiscriminative nature of PAXgene RNA stabilization reagent, the RNA content is dominated by RNA coming from the most abundant cellular species, that is erythrocytes. miRNA profiling using next generation sequencing (NGS) revealed that a typical human PAXgene peripheral blood RNA is dominated by several miRNAs and Y-RNA fragments which typically bear no diagnostic value and are unnecessarily consuming read capacity of an NGS instrument.

Here a method that can increase the read depth and discovery of the small RNA species in a given sample using an NGS pipeline by blocking the unwanted (abundant) RNA species is described. This method is applicable to any target RNA, any RNA samples and any library prep protocol that utilizes reverse transcription step.

The principle is to form an oligo-target RNA double stranded structure with a high melting temperature that inhibits, slowdowns, or halts, reverse transcriptase processivity. How exactly, on the molecular level, this occurs is currently unclear, as typically RTs have strand displacement activity. It is, however known in the field of RNA biochemistry, that highly structured, double stranded regions of RNA are unfavorable for the RT reaction, and eventually led to development of RT variants that can function at higher temperatures (up to 55° C.) in order to melt double stranded RNA structures.

### Experiment 1—qPCR and NGS for 3 Oligo Versions

To achieve a stable double stranded structure, the lock nucleic acid (LNA) modified nucleotides and/or ribonucleotides with 2′-o-methyl modifications were used as building blocks of the blocking oligonucleotides. To test the approach, miR-16-5p was targeted of which the mature canonical sequence is as follows:

(SEQ ID NO: 67)
5′-UAGCAGCACGUAAAUAUUGGCG-3′

whereas the underlined region is targeted by the antisense oligonucleotides block_miR-16-5p_v1, block_miR-16-5p_v2, and block_miR-16-5p_v3 (see below).

Three versions of antisense oligos against miR-16-5p were designed:

block_miR-16-5p_v1:
(SEQ ID NO: 68)
C+CAA+TA+TT+TA+CGT+GCT+GC
block_miR-16-5p_v2:
(SEQ ID NO: 69)
mCmCmAmAmUmAmUmUmUmAmCmGmUmGmCmUmGmC
block miR-16-5p_v3:
(SEQ ID NO: 2)
mCmCmAmA+TA+TT+TA+CG+TGmCmUmGmC

whereas the “+N” denotes an LNA nucleotide, and “mN” a 2′-o-methyl ribonucleotide.

Next, to test the efficacy of the blocking, following experiment was prepared.

100 ng of PAXgene RNA was used for 3′ adapter ligation reaction as outlined below.

| Component | Volume | |
|---|---|---|
| 10X T4 RNA Ligase Reaction Buffer (NEB) | 2 | μl |
| 20% PEG8000 (Jena Bioscience) | 10 | μl |
| PAXgene RNA (20 ng/μl) | 5 | μl |
| 5′ pre-adenylated DNA linker (5 μM) | 1 | μl |
| RNase inhibitor murine (NEB) | 1 | μl |
| T4 RNA Ligase 2 truncated KQ (200U/μl, NEB) | 1 | μl |

Pre-adenylated DNA linker sequence:
(SEQ ID NO: 70)
5′-AACTGTAGGCACCATCAAT-3′.

The reaction was held 1 hour at 25° C. followed by 15 min at 65° C. Then was the reaction cooled to 4° C. To the previous reaction, the following components were added for the ligation of 5′ adapter.

| Component | Volume | |
|---|---|---|
| Previous reaction | 20 | μl |
| 10X T4 RNA Ligase Reaction Buffer (NEB) | 2 | μl |
| ATP (10 mM) | 2 | μl |
| RNA Adapter (5 μM) | 1 | μl |
| RNase inhibitor murine (NEB) | 1 | μl |
| T4 RNA Ligase 1 (30U/μl, NEB) | 1 | μl |
| Nuclease-free water | 13 | μl |

RNA adapter sequence:
(SEQ ID NO: 71)
5′-GUUCAGAGUUCUACAGUCCGACGAUC-3′

The reaction was held 1 hour at 25° C. followed by 15 min at 65° C. Then was the reaction cooled to 4° C.

To the previous reaction, 1 μl of blocking oligo from following stock were added: 100 μM; 10 μM; 1 μM; 0.1 μM.

| | |
|---|---|
| Blocking oligonucleotide | 1 μl |

The reaction was denatured at 85° C. for 2 min and then ramped down at 0.1° C./second to 25° C. in a PCR machine and then holding at 25° C. for 5 min. Then the reaction was placed on ice and the following components were added:

| Component | Volume | |
|---|---|---|
| Previous reaction | 41 | μl |
| 5x Maxima RT buffer (Thermofisher Scientific) | 12 | μl |
| Maxima RT (Thermofisher Scientific) | 2 | μl |
| RNase inhibitor murine (NEB) | 1 | μl |
| RT primer (5 μM) | 1 | μl |
| Nuclease-free water | 3 | μl |

The sequence of the RT primer DNA oligo is as follows (where “+” denotes locked nucleic acid, N is any deoxyribonucleotide):

(SEQ ID NO: 72)
5′-AGACGTGTGCTCTTCCGATCTNNNNNNNNNNNNA
TTGATG+GT+GCCTACAGTT-3′
(“+” denotes LNA enhanced nucleotide,
the nucleotide to the right of “+” is
an LNA enhanced nucleotide, “N” denotes
a random deoxynucleotide).

The reaction was incubated at 50° C. for 30 min and then 5 min at 85° C. The reaction was then placed on ice.

Next, the cDNA was purified using Omega MagBind pure NGS beads (Omega Bio-Tek) as recommended by the vendor. 2.5× ratio of the beads to the cDNA reaction volume was used. The cDNA was eluted in 25 μl of 10 mM Tris-HCl, pH 8.5.

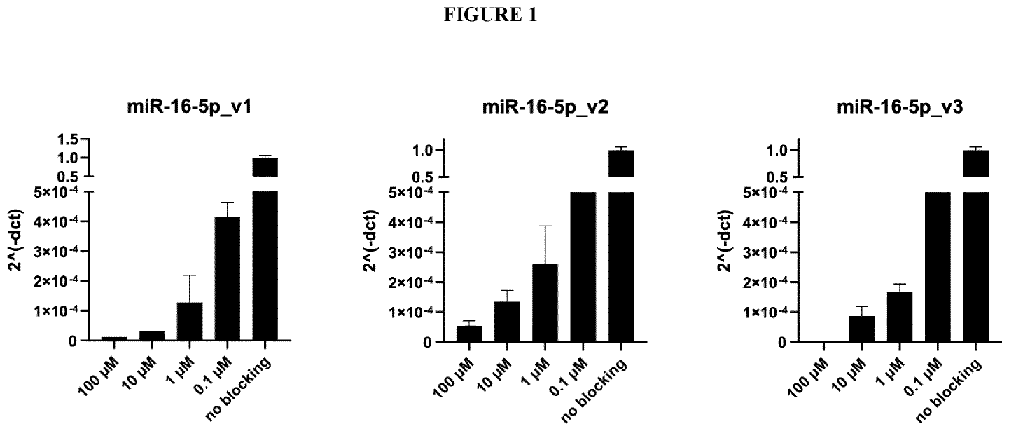

cDNA was subsequently diluted 1:25 with nuclease-free water and used for SyBr green based qPCR detection. The qPCR test (FIG. 1) indicated that the custom oligos can suppress the expression of miR-16-5p 10,000-fold or more.

Next, cDNA for the samples with 10 μM blocking oligos were used for library PCR using following components:

| Component | Volume | |
|---|---|---|
| Purified cDNA | 11 | μl |
| NEBNext ® Ultra ™ II Q5 ® Master Mix 2x | 12.5 | μl |
| NEB | | |
| Index primers (at 5 μM) | 1 | μl |
| Nuclease free water | 0.5 | μl |

The sequences of the indexing primers were as follows:

Forward primer:
(SEQ ID NO: 73)
AATGATACGGCGACCACCGAGATCTACACNNNNNNNNA
CACGTTCAGAGTTCTACAGTCCG*A.
Reverse primer:
(SEQ ID NO: 74)
CAAGCAGAAGACGGCATACGAGATATNNNNNNNNGACT
GGAGTTCAGACGTGTGCTCTTCCGATC*T.
(“*” denotes phosphortioate bond, while
“N” denotes 8 nucleotide index sequence
unique for each primer)

The reaction conditions were as follows:

| | | | | | |
|---|---|---|---|---|---|
| | Initial denaturation | 2 | min | 95° | C. |
| 18 | Denaturation | 15 | sec | 95° | C. |
| cycles | Annealing | 30 | sec | 60° | C. |
| | Extension | 15 | sec | 72° | C. |
| | Final extension | 2 | min | 72° | C. |

| | | | |
|---|---|---|---|
| Hold | at least 5 min | 4° | C. |

Next, the PCR product was purified using Omega MagBind pure NGS beads (Omega Bio-Tek) as recommended by the vendor. First, a 0.9× ratio of the beads to the PCR reaction volume was used and supernatant was used for second binding reaction with 1.8× ratio of beads to supernatant. The DNA was eluted in 20 μl of 10 mM Tris-HCl, pH 8.5.

Next, the DNA concentration was determined using QuantIT (ThermoFisher Scientific). Equimolar pool of DNA libraries was prepared and loaded as 500 μM concentration on NextSeq2000 P3 50 cycles cartridge (Illumina). Custom index 2 primer was used for sequencing (sequence: 5′-GATCGTCGGACTGTAGAACTCTGAACGTGT-3′ (SEQ ID NO: 75)).

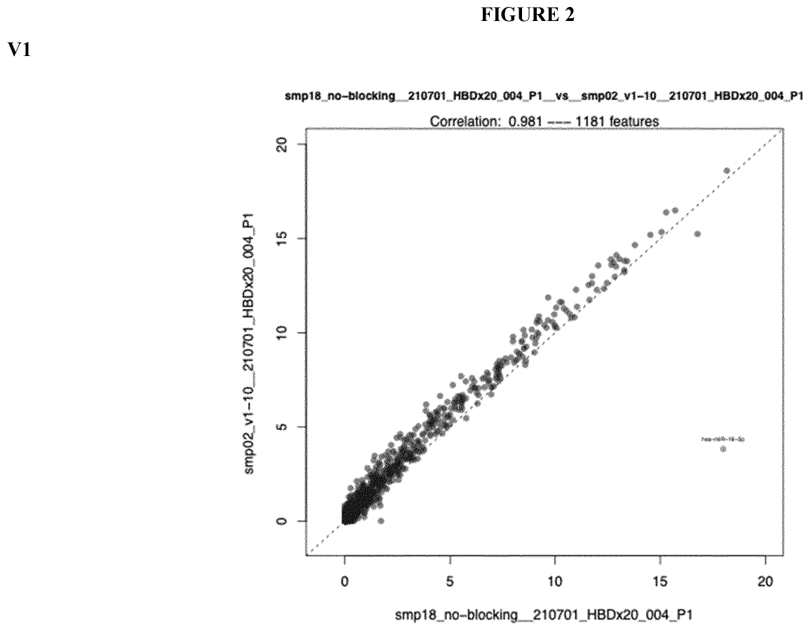

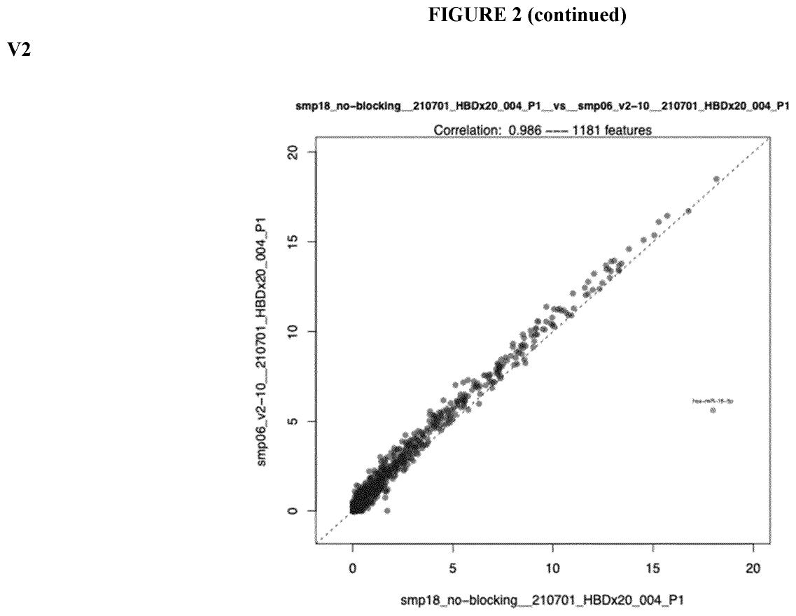

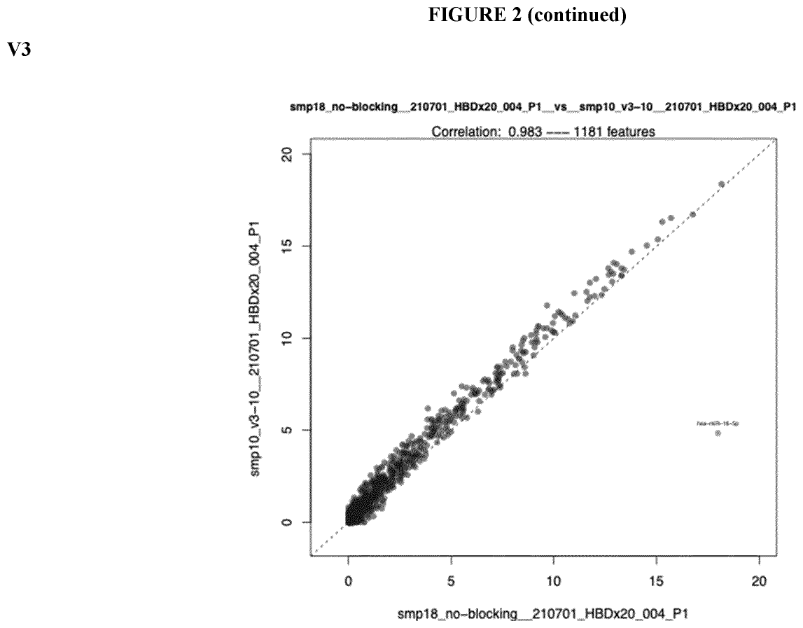

The sequence files were preprocessed by trimming the 3′adapter sequence. Read count matrix was established and data mapped to miR Base v22.0. RPM values for a blocked sample and unblocked control sample were calculated. FIG. 2 shows the results of the test with 10 μM concentration of each oligo and plots the RPM (reads per million, log 2) values of miR-16-5p in blocked sample versus unblocked control (annotated). Other miRNAs are colored in dark grey. While the blocking efficacy, measured by the decrease of RPM value of the miR-16-5p, is best achieved for the v1 of the oligo, there appears to be an off-target miRNA with high abundancy that is affected by the v1. v2 and v3 oligos do not appear to cause off-target effects on mapped miRNAs and their correlation coefficients are higher than the correlation coefficient for the v1 version.

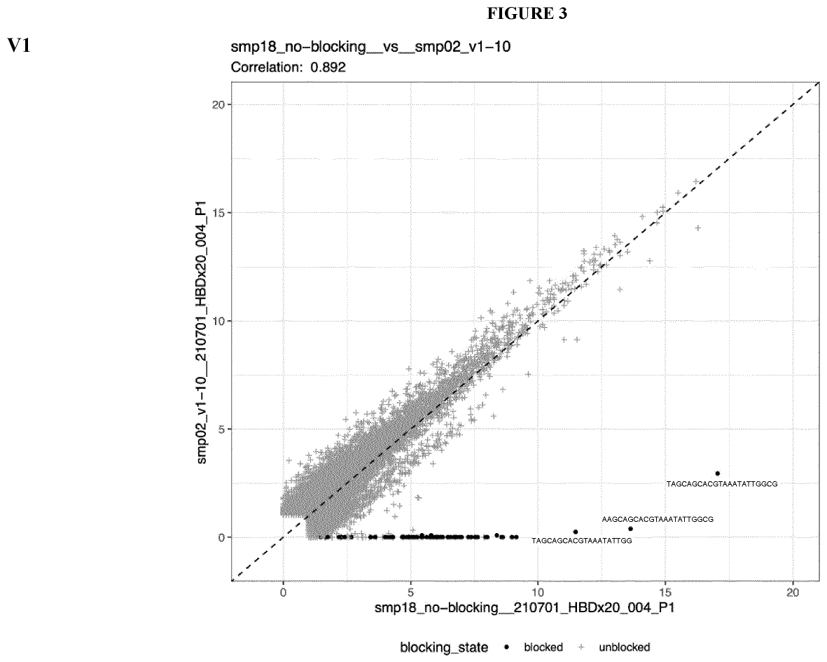

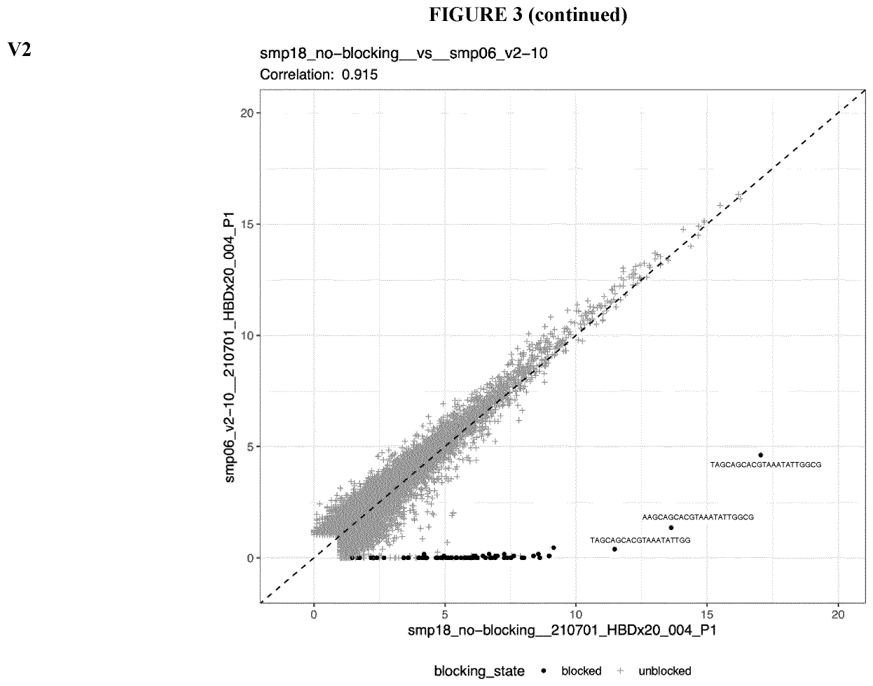

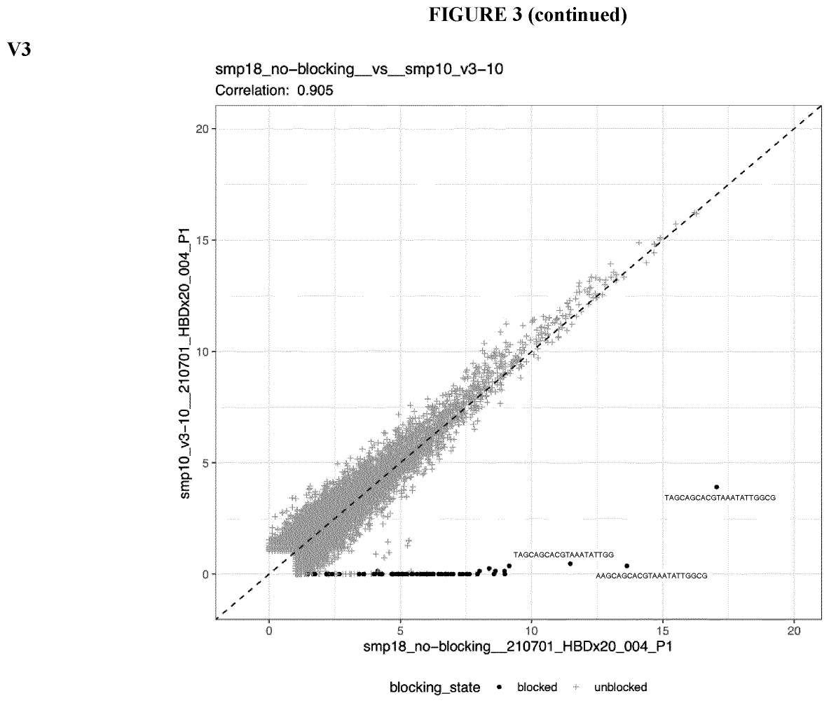

Next, the RPM values of all detected RNAs (not only canonical miRNAs) were inspected and the off-target effects and overall correlation of samples were evaluated (FIG. 3). Similarly, the version 2 and 3 of the blocking oligos show higher correlation and less off-target effect (as seen by grey dots appearing further away from the diagonal).

Taking the qPCR and NGS results into account, it was decided to continue to work with design version 3 (v3).

### Experiment 2—NGS Blocking 33 Target RNAs and Others

For the purpose of removing miRNAs/Y-RNAs from the NGS experiment by biochemical methods, the most abundant miRNA/Y-RNA species in PAXgene human blood RNA sample were defined:

| | | | Concentration of |
|---|---|---|---|
| | % pro | | block oligo in |
| Rank | reads consumed | Target | mixture (μM) |
| 1 | 17.2304 | hsa-miR-486-5p | 10.0 |
| 2 | 15.7410 | hsa-miR-16-5p | 5.0 |
| 3 | 13.0688 | hsa-miR-191-5p | 5.0 |
| 4 | 9.3483 | hY4 fragment | 5.0 |
| 5 | 3.9292 | hsa-miR-25-3p | 7.5 |
| 6 | 3.1562 | hsa-miR-92a-3p | 10.0 |
| 7 | 2.8768 | hsa-miR-223-3p | 1.0 |
| 8 | 1.5220 | hsa-let-7a-5p | 1.0 |
| 9 | 1.2423 | hsa-let-7i-5p | 2.5 |
| 10 | 1.2260 | hsa-miR-93-5p | 1.0 |
| 11 | 1.1550 | hsa-miR-142-5p | 1.0 |
| 12 | 0.9375 | hsa-miR-140-3p | 2.5 |
| 13 | 0.9300 | hsa-miR-30d-5p | 2.5 |
| 14 | 0.8333 | hsa-miR-182-5p | 2.5 |
| 15 | 0.8324 | hsa-miR-185-5p | 1.0 |
| 16 | 0.7683 | hsa-miR-103a-3p | 2.5 |
| 17 | 0.7347 | hsa-let-7b-5p | 1.0 |
| 18 | 0.6813 | hsa-miR-451a | 1.0 |
| 19 | 0.6783 | hsa-miR-150-5p | 2.5 |
| 20 | 0.5436 | hsa-miR-126-3p | 1.0 |
| 21 | 0.3993 | hsa-let-7f-5p | 1.0 |
| 22 | 0.3437 | hsa-miR-423-5p | 1.0 |
| 23 | 0.2804 | hY3 fragment | 1.0 |
| 24 | 0.2509 | hsa-miR-26a-5p | 1.0 |
| 25 | 0.2448 | hsa-miR-342-3p | 1.0 |
| 26 | 0.2266 | hsa-miR-128-3p | 1.0 |
| 27 | 0.2064 | hY1 fragment | 1.0 |
| 28 | 0.2001 | hsa-miR-425-5p | 5.0 |
| 29 | 0.1942 | hsa-miR-125b-5p | 1.0 |
| 30 | 0.1829 | hsa-miR-4732-3p | 1.0 |
| 31 | 0.1726 | hsa-miR-30c-5p | 2.5 |
| 32 | 0.1302 | hsa-let-7g-5p | 1.0 |
| 33 | 0.0525 | hsa-miR-484 | 1.0 |

Blocking oligos for each of these target miRNAs/Y-RNAs were designed and ordered from commercial vendor at RNase-free HPLC quality.

The blocking oligos designed and used can be taken from the following Table:

| SEQ ID | | |
|---|---|---|
| NO: | Oligo name | Oligo sequence (5′ to 3′) |
| 1 | block_miR-486-5p_v3a | mCmUmCmGmG+GG+CA+GC+T+CAmGmUmAmC |
| 2 | block_miR-16-5p_v3 | mCmCmAmA+TA+TT+TA+CG+TGmCmUmGmC |
| 3 | block_miR-191-5p_v3 | mGmCmUmGC+TT+TT+GG+GA+TmUmCmCmG |
| 4 | block_hY4_v3a | mCmAmGmUCA+AA+TT+TA+GCA+GmUmGmGmG |
| 5 | block_miR-25-3p_v3 | mCmAmGmA+CC+GA+GA+CA+AmGmUmGmC |
| 6 | block_miR-223-3p_v3 | mGmGmGmU+AT+TT+GA+CA+AmAmCmUmG |
| 7 | block_let-7a_v3 | mAmAmC+TA+TA+CAA+CCTACTAmCmCmUmC |
| 8 | block_let-7i_v3a | mAmAmCmA+GC+ACA+AA+CT+ACmUmAmCmC |
| 9 | block_miR-93-5p_v3 | mCmCmUmG+CA+CG+AA+CA+GCmAmCmUmU |
| 10 | block_miR-140-3p_v3 | mGmUmGmG+TT+CT+AC+CC+TmGmUmGmG |
| 11 | block_miR-30d-5p_v3 | mUmCmCmA+GT+CGGGG+AT+GT+TmUmAmCmA |
| 12 | block_miR-182-5p_v3 | mUmGmAmG+TT+CT+AC+CA+TmUmGmCmC |
| 13 | block_miR-185-5p_v3 | mCmAmGmGAAC+TG+CC+TT+TC+TmCmUmCmC |
| 14 | block_miR-103a-3p_v3 | mCmAmUmAG+CC+CTG+TA+CA+AmUmGmCmU |
| 15 | block_let-7b_v3 | mAmAmC+CA+CA+CAA+CCTACTAmCmCmUmC |
| 16 | block_miR-451a_v3 | mAmCmUmCmAG+TA+AT+GG+TA+AmCmGmGmU |
| 17 | block_miR-150-5p_v3 | mAmCmUmGmGT+AC+AA+GG+GT+TmGmGmGmA |
| 18 | block_miR-126-3p_v3 | mCmGmCmA+TT+AT+TA+CT+CmAmCmGmG |
| 19 | block_let-7f_v3 | mAmAmC+TA+TAC+AA+TCTACTAmCmCmUmC |
| 20 | block_miR-423-5p_v3 | mAmGmUmCmU+CG+CT+CT+CT+GmCmCmC |
| 21 | block_hY3_v3 | mCmUmAmGmUCAAG+TG+AA+GC+AG+TmGmGmGmA |
| 22 | block_miR-26a-5p_v3 | mCmCmUmAT+CC+TG+GA+TT+AmCmUmUmG |
| 23 | block_miR-342-3p_v3 | mGmGmGmUG+CG+AT+TT+CT+GmUmGmUmG |
| 24 | block_miR-128-3p_v3 | mAmGmAmGA+CC+GG+TT+CA+CmUmGmUmG |
| 25 | block_hY1_v3 | mCmUmAmGmUCAAG+TG+CA+GT+AG+TmGmAmGmA |
| 26 | block_miR-425-5p_v3 | mAmAmCmGGGA+GT+GA+TC+GT+GmUmCmAmU |
| 27 | block_miR-125b-5p_v3 | mCmAmCmAAGT+TA+GG+GT+CT+CmAmGmGmG |
| 28 | block_miR-4732-3p_v3 | mAmGmAmACA+GG+AC+AG+GT+CmAmGmGmG |
| 29 | block_miR-30c-5p_v3 | mCmUmGmAGAGT+GT+AG+GAT+GT+TmUmAmCmA |
| 30 | block_let-7g_v3 | mAmCmUmG+TA+CAAA+CT+AC+TAmCmCmUmC |
| 31 | block_miR-484_v3 | mUmCmGmGG+AGG+GG+AC+TGAGmCmCmUmG |
| 32 | block_miR-92a-3p_v3b | mAmCmAmG+GCCG+GG+AC+AmAmGmUmGmC |
| 33 | block_miR-142-5p_v3b | mAmGmUmGC+TT+TC+TA+CT+TmUmAmUmG |

(whereas the “+N” denotes an LNA nucleotide, and “mN” denotes a 2′-o-methyl ribonucleotide)

The oligos having a nucleotide sequence according to SEQ ID NO:1 to SEQ ID NO: 33 are designed to bind to unwanted miRNAs and/or isomiRs or unwanted Y-RNAs and/or fragments thereof. They are summarized in the following Table:

| SEQ ID | target miRNA or Y- | Partial RNA seq of target |
|---|---|---|
| NO: | RNA canonical name | miRNA or Y-RNA (5′ to 3′) |
| 34 | hsa-miR-486-5p | GUACUGAGCUGCCCCGAG |
| 35 | hsa-miR-16-5p | GCAGCACGUAAAUAUUGG |
| 36 | hsa-miR-191-5p | CGGAAUCCCAAAAGCAGC |
| 37 | hY4 | CCCACUGCUAAAUUUGACUG |
| 38 | hsa-miR-25-3p | GCACUUGUCUCGGUCUG |
| 39 | hsa-miR-223-3p | CAGUUUGUCAAAUACCC |
| 40 | hsa-let-7a-5p | GAGGUAGUAGGUUGUAUAGUU |
| 41 | hsa-let-7i-5p | GGUAGUAGUUUGUGCUGUU |
| 42 | hsa-miR-93-5p | AAGUGCUGUUCGUGCAGG |
| 43 | hsa-miR-140-3p | CCACAGGGUAGAACCAC |
| 44 | hsa-miR-30d-5p | UGUAAACAUCCCCGACUGGA |
| 45 | hsa-miR-182-5p | GGCAAUGGUAGAACUCA |
| 46 | hsa-miR-185-5p | GGAGAGAAAGGCAGUUCCUG |
| 47 | hsa-miR-103a-3p | AGCAUUGUACAGGGCUAUG |
| 48 | hsa-let-7b-5p | GAGGUAGUAGGUUGUGUGGUU |
| 49 | hsa-miR-451a | ACCGUUACCAUUACUGAGU |
| 50 | hsa-miR-150-5p | UCCCAACCCUUGUACCAGU |
| 51 | hsa-miR-126-3p | CCGUGAGUAAUAAUGCG |
| 52 | hsa-let-7f | GAGGUAGUAGAUUGUAUAGUU |
| 53 | hsa-miR-423-5p | GGGCAGAGAGCGAGACU |
| 54 | hY3 | UCCCACUGCUUCACUUGACUAG |
| 55 | hsa-miR-26a-5p | CAAGUAAUCCAGGAUAGG |
| 56 | hsa-miR-342-3p | CACACAGAAAUCGCACCC |
| 57 | hsa-miR-128-3p | CACAGUGAACCGGUCUCU |
| 58 | hY1 | UCUCACUACUGCACUUGACUAG |
| 59 | hsa-miR-425-5p | AUGACACGAUCACUCCCGUU |
| 60 | hsa-miR-125b-5p | CCCUGAGACCCUAACUUGUG |
| 61 | hsa-miR-4732-3p | CCCUGACCUGUCCUGUUCU |
| 62 | hsa-miR-30c-5p | UGUAAACAUCCUACACUCUCAG |
| 63 | hsa-let-7g-5p | GAGGUAGUAGUUUGUACAGU |
| 64 | hsa-miR-484 | CAGGCUCAGUCCCCUCCCGA |
| 65 | hsa-miR-92a-3p | GCACUUGUCCCGGCCUGU |
| 66 | hsa-miR-142-5p | CAUAAAGUAGAAAGCACU |

Please note that only the RNA molecule region is indicated to which the oligos bind.

Additional oligos were designed. These oligos have a nucleotide sequence according to SEQ TD NO: 76 to SEQ ID NO: 122 and were designed to bind to other unwanted RNA species. Said oligos are summarized in the following Table:

| SEQ ID | | |
|---|---|---|
| NO: | Oligo name | Oligo sequence (5′ to 3′) |
| 76 | block_18S_1_v3 | mCmUmGmGC+AGG+ATC+AAC+CAmGmGmUmA |
| 77 | block_28S_2_v3 | mCmAmCmGT+CTG+ATC+TGA+GGmUmCmGmC |
| 78 | block_28S_9891_v3 | mAmCmUmUC+CAT+GGC+CAC+CGmUmCmCmU |
| 79 | block_rRNA_v3 | mAmAmCmCCTTGT+GT+CG+AG+GG+CmUmGmAmC |
| 80 | block_5.8S_v3 | mGmUmGmAT+CCA+CCG+CTA+AGmAmGmUmC |
| 81 | block_5S_v3a | mAmGmAmCG+AGA+TCGGGC+GCmGmUmUmC |
| 82 | block_5S_v3b | mGmGmGmUG+GTA+TGG+CCG+TmAmGmAmC |
| 83 | block_5S_v3c | mCmCmCmUG+CTT+AGC+TTC+CGmAmGmAmU |
| 84 | block_5S_v3d | mCmCmCmAT+CCA+AGT+ACT+AAmCmCmAmG |
| 85 | block_hY1_v3a | mAmGmAmGT+AGA+ACA+AGG+AGmUmUmCmG |
| 86 | block_hY1_v3b | mAmCmUmGT+GAA+CAA+TCA+ATmUmGmAmG |
| 87 | block_hY1_v3c | mUmCmAmCT+ACC+TTC+GGA+CCmAmGmCmC |
| 88 | block_hY3_v3a | mCmCmAmCT+GCA+CTC+GGA+CCmAmGmCmC |
| 89 | block_hY4_v3 | mAmGmUmCA+AA+TT+TA+GC+AmGmUmGmG |
| 90 | block_hY4_v3b | mGmUmUmGT+ATA+CCA+ACT+TTmAmGmUmG |
| 91 | block_hY4_v3c | mCmUmAmAT+GTT+AAT+AAG+TTmCmUmGmA |
| 92 | block_hY4_v3d | mCmCmAmCT+ACC+ATC+GGA+CCmAmGmCmC |
| 93 | block_hY5_v3a | mUmAmGmUC+AAG+CGC+GGT+TGmUmGmGmG |
| 94 | block_hY5_v3b | mCmCmAmCA+ACA+CTC+GGA+CCmAmAmCmU |
| 95 | block_snoRD12B_v3 | mAmAmAmGC+TAA+GTC+ATC+ATmAmUmAmU |
| 96 | block_snorD18A_v3 | mAmAmGmUG+GAA+TTT+CAT+CAmCmUmAmC |
| 97 | block_snoRD2_v3 | mAmGmAmUG+ATT+GCC+ATC+ATmUmUmCmA |
| 98 | block_snoRD26_v3 | mUmUmCmGT+AAA+ATC+ATC+CCmCmGmUmA |
| 99 | block_snorD27_v3 | mAmUmUmUT+GTG+TTC+ATC+ATmGmGmAmG |
| 100 | block_snoRD29_v3 | mUmAmGmUT+TGA+TTC+ATC+ATmAmGmAmA |
| 101 | block_snoRD30_v3 | mCmCmAmUG+TAA+GTC+ATC+ACmAmAmAmC |
| 102 | block_snoRD38B_v3 | mAmCmAmAA+GTT+TTC+ATC+ACmUmGmAmG |
| 103 | block_snoRD42B_v3 | mAmAmAmAC+TT+TT+CC+ATC+ATmAmUmGmC |
| 104 | block_snoRD43_v3 | mUmCmAmAT+AAG+TTC+ATC+ATmCmUmGmU |
| 105 | block_snoRD44_v3 | mUmUmUmGC+TTA+TCA+TCA+TmCmCmAmG |
| 106 | block_snoRD49A_v3 | mUmUmAmGT+GAT+TTC+ATC+AGmAmGmCmA |
| 107 | block_snoRD59B_v3 | mGmUmCmAG+AAC+GTA+CTC+ATCmAmGmUmG |
| 108 | block_snoRD6_v3 | mUmUmUmCG+CCC+ATC+ATC+ATmAmAmCmA |
| 109 | block_snoRD63_v3 | mGmAmAmUA+AAA+TA+CAT+CA+TmUmGmCmA |
| 110 | block_snoRD69_v3 | mAmUmCmCA+GTT+TAT+CAT+CAmUmUmUmG |
| 111 | block_snoRD76_v3 | mAmAmUmAA+ACT+GTC+ATC+ATmUmGmUmG |
| 112 | block_snoRD80_v3 | mAmAmCmUA+TGT+TAT+CAT+CAmUmUmGmU |
| 113 | block_snoRD81_v3 | mGmGmAmUT+GAG+ATC+ATC+ATmGmUmAmU |
| 114 | block_tRNA_Ala_v3 | mUmGmGmU+G+GA+GA+TG+CmCmGmGmG |
| 115 | block_tRNAAla_v3a | mCmUmAmCC+ATT+TGA+GCT+AAmUmCmCmC |
| 116 | block_tRNAArg_v3 | mUmAmUmCC+ATT+GCG+CCA+CGmGmAmGmC |
| 117 | block_tRNAGlu_v3 | mUmAmAmCC+ACT+AGA+CCA+CCmAmGmGmG |
| 118 | block_tRNALeu_v3 | mGmAmCmCG+CTC+GGC+CAT+CCmUmGmAmC |
| 119 | block_tRNALys_v3 | mCmUmAmCC+GAC+TGA+GCT+AGmCmCmGmG |
| 120 | block_tRNASeCys_v3 | mAmCmCmAC+TGA+GGA+TCA+TCmCmGmGmG |
| 121 | block_tRNASer_v3 | mAmAmCmCA+CTC+GGC+CAC+GAmCmUmAmC |
| 122 | block_tRNAVal_v3 | mAmAmCmCA+CTA+CAC+TAC+GGmAmAmAmC |

(whereas the “+N” denotes an LNA nucleotide, and “mN” denotes a 2′-o-methyl ribonucleotide)

The unwanted RNA species to which the oligos having a nucleotide sequence according to SEQ ID NO: 76 to SEQ ID NO: 122 bind are summarized in the following Table:

| SEQ | target RNA | partial RNA |
|---|---|---|
| ID | canonical | sequence of |
| NO: | name | target RNA (5′ to 3′) |
| 123 | hsa-18S | UACCUGGUUGAUCCUGCCAG |
| 124 | hsa-28S | GCGACCUCAGAUCAGACGUG |
| 125 | hsa-28S | AGGACGGUGGCCAUGGAAGU |
| 126 | hsa-28S | GUCAGCCCUCGACACAAGGGUU |
| 127 | hsa-5.8S | GACUCUUAGCGGUGGAUCAC |
| 128 | hsa-5S | GAACGCGCCCGAUCUCGUCU |
| 129 | hsa-5S | GUCUACGGCCAUACCACCC |
| 130 | hsa-5S | AUCUCGGAAGCUAAGCAGGG |
| 131 | hsa-5S | CUGGUUAGUACUUGGAUGGG |
| 132 | hsa-hY1 | CGAACUCCUUGUUCUACUCU |
| 133 | hsa-hY1 | CUCAAUUGAUUGUUCACAGU |
| 134 | hsa-hY1 | GGCUGGUCCGAAGGUAGUGA |
| 135 | hsa-hY3 | GGCUGGUCCGAGUGCAGUGG |
| 136 | hsa-hY4 | CCACUGCUAAAUUUGACU |
| 137 | hsa-hY4 | CACUAAAGUUGGUAUACAAC |
| 138 | hsa-hY4 | UCAGAACUUAUUAACAUUAG |
| 139 | hsa-hY4 | GGCUGGUCCGAUGGUAGUGG |
| 140 | hsa-hY5 | CCCACAACCGCGCUUGACUA |
| 141 | hsa-hY5 | AGUUGGUCCGAGUGUUGUGG |
| 142 | hsa-snoRD12B | AUAUAUGAUGACUUAGCUUU |
| 143 | hsa-snorD18A | GUAGUGAUGAAAUUCCACUU |
| 144 | hsa-snoRD2 | UGAAAUGAUGGCAAUCAUCU |
| 145 | hsa-snoRD26 | UACGGGGAUGAUUUUACGAA |
| 146 | hsa-snorD27 | CUCCAUGAUGAACACAAAAU |
| 147 | hsa-snoRD29 | UUCUAUGAUGAAUCAAACUA |
| 148 | hsa-snoRD30 | GUUUGUGAUGACUUACAUGG |
| 149 | hsa-snoRD38B | CUCAGUGAUGAAAACUUUGU |
| 150 | hsa-snoRD42B | GCAUAUGAUGGAAAAGUUUU |
| 151 | hsa-snoRD43 | ACAGAUGAUGAACUUAUUGA |
| 152 | hsa-snoRD44 | CUGGAUGAUGAUAAGCAAA |
| 153 | hsa-snoRD49A | UGCUCUGAUGAAAUCACUAA |
| 154 | hsa-snoRD59B | CACUGAUGAGUACGUUCUGAC |
| 155 | hsa-snoRD6 | UGUUAUGAUGAUGGGCGAAA |
| 156 | hsa-snoRD63 | UGCAAUGAUGUAUUUUAUUC |
| 157 | hsa-snoRD69 | CAAAUGAUGAUAAACUGGAU |
| 158 | hsa-snoRD76 | CACAAUGAUGACAGUUUAUU |
| 159 | hsa-snoRD80 | ACAAUGAUGAUAACAUAGUU |
| 160 | hsa-snoRD81 | AUACAUGAUGAUCUCAAUCC |
| 161 | hsa-tRNAAla | CCCGGCAUCUCCACCA |
| 162 | hsa-tRNAAla | GGGAUUAGCUCAAAUGGUAG |
| 163 | hsa-tRNAArg | GCUCCGUGGCGCAAUGGAUA |
| 164 | hsa-tRNAGlu | CCCUGGUGGUCUAGUGGUUA |
| 165 | hsa-tRNALeu | GUCAGGAUGGCCGAGCGGUC |
| 166 | hsa-tRNALys | CCGGCUAGCUCAGUCGGUAG |
| 167 | hsa-tRNASeCys | CCCGGAUGAUCCUCAGUGGU |
| 168 | hsa-tRNASer | GUAGUCGUGGCCGAGUGGUU |
| 169 | hsa-tRNA Val | GUUUCCGUAGUGUAGUGGUU |

Please note that only the RNA molecule region is indicated to which the oligos bind.

### Oligo Mixture

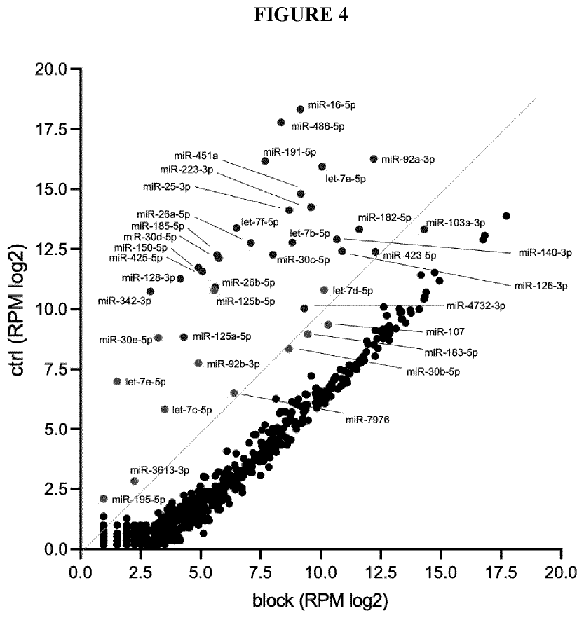

Dried oligos were resuspended in RNase free water and mixed together in water with final concentration as outlined in the table above. The oligo mixture was stored at −20° C. The oligo mixture was used in an NGS experiment similarly as described above. FIG. 4 shows a result of such experiment, in which blocked miRNA species appear close or above the diagonal, while unblocked miRNA species appear further below. Unblocked miRNAs exhibit increase in their RPMs. miRNAs that showed blocking effect were annotated.

1. A blocking oligonucleotide comprising one or more modified ribonucleotides and/or one or more locked nucleotides.

2. The blocking oligonucleotide of claim 1, wherein the blocking oligonucleotide is reverse complementary to at least a region of an unwanted RNA molecule and wherein the unwanted RNA molecule is an abundant non-coding RNA molecule having a length of <200 ribonucleotides.

3.-4. (canceled)

5. The blocking oligonucleotide of claim 2, wherein the unwanted RNA molecule is an abundant miRNA, miRNA isoform (isomiR), Y-RNA, Y-RNA fragment, that is not a full-length Y-RNA, snoRNA, rRNA fragment, that is not full-length rRNA, or tRNA.

6. The blocking oligonucleotide of claim 1, wherein
the one or more modified ribonucleotides are comprised in the 5′terminal nucleotide sequence and/or in the 3′terminal nucleotide sequence of the blocking oligonucleotide, and/or
the one or more locked nucleotides are comprised in the central nucleotide sequence of the blocking oligonucleotide.

7. (canceled)

8. The blocking oligonucleotide of claim 1, wherein the blocking oligonucleotide comprises in the following order from 5′ to 3′:
(i) a 5′terminal nucleotide sequence comprising one or more (consecutive) modified ribonucleotides,
(ii) a central nucleotide sequence comprising one or more (non-consecutive) locked nucleotides, and
(ii) a 3′terminal nucleotide sequence comprising one or more (consecutive) modified ribonucleotides.

9.-10. (canceled)

11. The blocking oligonucleotide of claim 1, wherein the blocking oligonucleotide comprises between 3 and 10 locked nucleotides.

12. (canceled)

13. The blocking oligonucleotide of claim 1, wherein the blocking oligonucleotide comprises between 4 and 22 modified ribonucleotides.

14. The blocking oligonucleotide of claim 1, wherein the modified ribonucleotide(s) is (are) 2′-o-methyl ribonucleotide(s).

15. The blocking oligonucleotide of claim 1, wherein the locked nucleotide(s) is (are) selected from the group consisting of LNA-adenine, LNA-guanine, LNA-cytosine, and LNA-thymine.

16-18. (canceled)

19. The blocking oligonucleotide of claim 1, wherein the blocking oligonucleotide is reverse complementary to at least a region of an unwanted RNA molecule having a nucleotide sequence selected from the group consisting of SEQ ID NO: 34 to SEQ ID NO: 66 and SEQ ID NO: 123 to SEQ ID NO: 169.

20. The blocking oligonucleotide of claim 1, wherein the blocking oligonucleotide has a nucleotide sequence selected from the group consisting of SEQ ID NO: 1 to SEQ ID NO: 33 and SEQ ID NO: 76 to SEQ ID NO: 122.

21. A set comprising at least two blocking oligonucleotides of claim 1.

22. The set of claim 21, wherein
the at least two blocking oligonucleotides differ from each other in their nucleotide sequence, and/or
the at least two blocking oligonucleotides are reverse complementary to at least a region of different unwanted RNA molecules.

23. (canceled)

24. A method for inhibiting cDNA synthesis of one or more unwanted RNA molecules in an RNA sample during reverse transcription comprising the steps of:
(i) providing a mixture containing an RNA sample comprising one or more desired RNA molecules and one or more unwanted RNA molecules and one or more blocking oligonucleotides of claim 1, wherein the one or more blocking oligonucleotides are annealed to the one or more unwanted RNA molecules, and
(ii) reverse transcribing the one or more desired RNA molecules, thereby obtaining one or more cDNA products from said desired RNA molecule(s) and inhibiting cDNA synthesis of the one or more unwanted RNA molecules.

25. A method for (improving) cDNA synthesis of one or more desired RNA molecules in an RNA sample during reverse transcription comprising the steps of:
(i) providing a mixture containing an RNA sample comprising one or more desired RNA molecules and one or more unwanted RNA molecules and one or more blocking oligonucleotides of claim 1, wherein the one or more blocking oligonucleotides are annealed to the one or more unwanted RNA molecules, and
(ii) reverse transcribing the one or more desired RNA molecules, thereby obtaining one or more cDNA products from said desired RNA molecule(s) and inhibiting cDNA synthesis of the one or more unwanted RNA molecules.

26-38. (canceled)

39. A method for producing one or more double stranded cDNA products from one or more desired RNA molecules comprising the steps of:
(i) carrying out the method of claim 24, and
(ii) amplifying the one or more cDNA products, thereby producing one or more double stranded cDNA products from one or more desired RNA molecules.

40-44. (canceled)

45. A method for determining a profile of one or more desired RNA molecules comprising the step of:
sequencing the one or more double stranded cDNA products from the one or more desired RNA molecules produced by the method of claim 39.

46-49. (canceled)

50. A kit comprising one or more of the oligonucleotide of claim 1.

51-63. (canceled)

64. A method for producing one or more double stranded cDNA products from one or more desired RNA molecules comprising the steps of:
(i) carrying out the method of claim 25, and
(ii) amplifying the one or more cDNA products, thereby producing one or more double stranded cDNA products from one or more desired RNA molecules.

65. A method for determining a profile of one or more desired RNA molecules comprising the step of:
sequencing the one or more double stranded cDNA products from the one or more desired RNA molecules produced by the method of claim 64.
